(19) United States
(12) Patent Application Publication
(10) Pub. No.: US 2026/0295091 A1
(43) Pub. Date: Oct. 1, 2026

(54) TREATMENT OF MULTIPLE SCLEROSIS USING NT-3 GENE THERAPY

(71) Applicant: RESEARCH INSTITUTE AT NATIONWIDE CHILDREN'S HOSPITAL, Columbus, OH (US)

(72) Inventor: Zarife Sahenk, Columbus, OH (US)

(73) Assignee: RESEARCH INSTITUTE AT NATIONWIDE CHILDREN'S HOSPITAL, Columbus, OH (US)

(21) Appl. No.: 19/480,332

(22) PCT Filed: May 1, 2024
(86) PCT No.: PCT/US2024/027246

§ 371 Date: Oct. 30, 2025

Related U.S. Application Data
(60) Provisional application No. 63/463,501, filed on May 2, 2023.

(51) Int. Cl. A61K 48/00; A61K 9/00; A61K 38/18; A61P 37/06; C12N 15/86

(52) U.S. Cl. CPC A61K 48/0058; A61K 9/0019; A61K 38/185; A61K 48/0075; A61P 37/06; C12N 15/86; C12N 2750/14143; C12N 2830/008; C12N 2830/42

(57) ABSTRACT

The present disclosure relates to methods of treating an autoimmune disease, such as multiple sclerosis, using the rAAV expressing NT-3 gene therapy.

### CROSS REFERENCE TO RELATED APPLCIATIONS

This application claims priority benefit of U.S. Provisional Application No. 63/463,501, filed on May 2, 2023, which is incorporated herein by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 59101_SeqListing.xml; Size: 23,953 bytes; Created: May 1, 2024.

### FIELD

The present disclosure relates to methods of using the rAAV expressing NT-3 for gene therapy to treat multiple sclerosis. The present disclosure also relates to methods of using the rAAV expressing NT-3 for gene therapy to treat autoimmune disease.

### BACKGROUND

Multiple sclerosis (MS) is a common autoimmune demyelinating disease affecting more than 2.5 million patients worldwide. MS, also known as encephalomyelitis disseminate, is an autoimmune disease of which the patients' immune system attacks the myelin of the nerves in brain and spinal cord, and eventually causes demyelination of the nerves leading to secondary axonal loss. Unfortunately, the main causes of MS remain largely unknown, and currently there is no cure for the disease.

Autoimmune disease is a condition arising from an abnormal immune response to a functioning body part, such as a cell, tissue and/or organ. At least 80 types of autoimmune diseases have been identified, with some evidence suggesting that there may be more than 100 types. Nearly any body part can be involved. Common symptoms can be diverse and transient, ranging from mild to severe, and generally include low grade fever and feeling tired. Similar to MS, the cause of autoimmune disease is unknown. Some autoimmune diseases such as lupus run in families, and certain cases may be triggered by infections or other environmental factors. Treatment depends on the type and severity of the condition. Nonsteroidal anti-inflammatory drugs (NSAIDs) and immunosuppressants are often used. Intravenous immunoglobulin may also occasionally be used. While these treatments usually improve symptoms, they do not typically cure the disease.

Recent studies have demonstrated that neurotrophin 3 (NT-3) is a versatile molecule with previously unknown or underappreciated features. In addition to its well-recognized effects on peripheral nerve regeneration and Schwan cells (SCs), NT-3 has anti-inflammatory and immunomodulatory effects (Yang et al., Mel Titer, 22 (2): 440-450 (2014)). It has been recently demonstrated that NT-3 is capable of attenuating spontaneous autoimmune peripheral polyneuropathy in the rodent model of chronic inflammatory demyelinating peripheral nerve disorder that occurs in humans (Yalvac et al., Gene therapy, 23 (1): 95-102 (2015)).

NT-3 is a trophic factor secreted by Schwann cells (SCs) that supports nerve regeneration. The ability of denervated SCs to survive is crucial for nerve regeneration as SCs provide both growth factors and basal lamina, scaffoldings that promote axonal growth. Prolonged denervation leads to a decreased regenerative capacity related to reduced expression of regeneration-associated SC molecules (neurotrophic factors (NTFs) and their receptors) resulting in atrophy of the denervated SCs, breakdown of the bands of Bungner, and loss of SC basal lamina scaffoldings.

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter, and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Accordingly, there is a need for developing therapies for autoimmune diseases such as multiple sclerosis. The disclosure provides gene therapy methods for delivering NT-3 for the treatment of multiple sclerosis and other autoimmune disease.

### SUMMARY

The disclosure provides methods of treating autoimmune multiple sclerosis. The method comprises administering a therapeutically effective amount of neurotrophin-3 (NT-3 or NTF-3; these terms are used interchangeably in this application), pro-NT-3, or an effective fragment thereof, or a nucleic acid encoding NT-3, pro-NT-3, or an effective fragment thereof, to a subject with multiple sclerosis or other autoimmune disease.

NT-3, like other neurotrophins, was initially identified because of its essential function in nervous system development, myelination, growth, axonal protection, and regeneration control (12-16). However, broader effects of NT-3 are now recognized, extending to a wide range of cell types, including immune cells and participation in inflammatory responses. Studies suggest that NT-3 plays a critical role in the regulation or maintenance of the T helper cell 1 and 2 (Th1/Th2) balance through interaction with its receptor, TrkC, expressed by Th2 cells (17). NT-3 acts as a trigger for IL-4 production in TrkC-expressing Th2 cells, affecting Dendritic cell (DC) maturation and inducing regulatory DC formation. Thus, the NT-3/TrkC system is thought to be involved in the induction or maintenance of Th2-dependent immunity. The efficacy of the immunomodulatory and anti-inflammatory properties of NT-3 was previously demonstrated in the spontaneous autoimmune peripheral polyneuropathy (SAPP) mouse model (18) for chronic inflammatory polyradiculopathy (CIDP) in humans, using the AAV1.NT-3 gene therapy approach (19-23). This approach was developed due to the short half-life of NT3 peptide in serum, requiring repeated subcutaneous delivery (24). In contrast, a gene therapy approach entails intramuscular (IM) delivery of the scAAV1.tMCK.NT-3 vector, providing a systemic effect following transduction of muscle to produce NT-3 protein, which is released into serum continuously, as detected by ELISA (18-23). The treated SAAP mice showed increased hindlimb grip strength, correlating with improved compound muscle action potential, CMAP and increased remyelinated nerve fiber population, along with decreased number of infiltrating CD3+T cells, as well as reduced expression of tumor necrosis factor (TNF)-α and interleukin IL-1β, with an increase of IL-10 and FoxP3 in sciatic nerves (18). In addition, bone marrow-derived DCs, when challenged with bacterial lipopolysaccharide (LPS) in the presence of NT-3, showed significantly increased IL-10 secretion and decreased TNF-α, indicating that DCs gained tolerogenic features (18). The anti-inflammatory effect of NT-3 was previously shown in the EAE model through the creation of an anti-inflammatory cytokine milieu in the spinal cord by NT-3-transduced embryonic stem cell-derived microglia (25). Moreover, NT-3 promotes oligodendrocyte precursor proliferation, survival, and differentiation, and myelin protein synthesis (26, 27). Furthermore, NT-3 has significant capacity to provide neuroprotection and reduce astrogliosis (28), which is important in the formation of MS plaque. Therefore, success in neuroprotection and immunomodulation by NT-3 potentially fulfills the requirements necessary for the treatment of EAE and its clinical correlate chronic progressive MS.

The disclosure provides for a method of treating an autoimmune disease in a human subject in need thereof comprising the step of administering to the human subject a nucleic acid encoding a NT-3 polypeptide; wherein a) the nucleic acid comprises a nucleotide sequence that is at least 90% identical to the nucleotide sequence of SEQ ID NO: 1; b) the nucleic acid comprises the nucleotide sequence of SEQ ID NO: 1; c) the nucleic acid comprises a nucleotide sequence encoding an amino acid sequence that is at least 90% identical to SEQ ID NO: 2; or d) the nucleic acid comprises a nucleotide sequence encoding the amino acid sequence of SEQ ID NO: 2. In some embodiments, the autoimmune disease is Alopecia areata, Addison disease, Celiac disease, Crohn's disease, Ulcerative colitis, Autoimmune inflammatory myositis, Graves disease, Hashimoto thyroiditis, inflammatory bowel disease, Multiple sclerosis, Pemphigus, Pernicious anemia, Psoriasis, Reactive arthritis, Rheumatoid arthritis, Sjögren syndrome, Systemic lupus erythematosus, Type I diabetes, or Autoimmune hepatitis.

In one embodiment, the disclosure provides for a method of treating multiple sclerosis in a human subject in need thereof comprising the step of administering to the human subject a nucleic acid encoding a NT-3 polypeptide; wherein a) the nucleic acid comprises a nucleotide sequence that is at least 90% identical to the nucleotide sequence of SEQ ID NO: 1; b) the nucleic acid comprises the nucleotide sequence of SEQ ID NO: 1; c) the nucleic acid comprises a nucleotide sequence encoding an amino acid sequence that is at least 90% identical to SEQ ID NO: 2; or d) the nucleic acid comprises a nucleotide sequence encoding the amino acid sequence of SEQ ID NO: 2.

The disclosure provides for a nucleic acid encoding a NT-3 polypeptide or a composition comprising said nucleic acid for use in treating an autoimmune disease in a human subject in need thereof wherein a) the nucleic acid comprises a nucleotide sequence that is at least 90% identical to the nucleotide sequence of SEQ ID NO: 1; b) the nucleic acid comprises the nucleotide sequence of SEQ ID NO: 1; c) the nucleic acid comprises a nucleotide sequence encoding an amino acid sequence that is at least 90% identical to SEQ ID NO: 2; or d) the nucleic acid comprises a nucleotide sequence encoding the amino acid sequence of SEQ ID NO: 2. For example, any of the disclosed viral vectors are useful for treating autoimmune disease such as Alopecia areata, Addison disease, Celiac disease, Crohn's disease, Ulcerative colitis, Autoimmune inflammatory myositis, Graves disease, Hashimoto thyroiditis, inflammatory bowel disease, Multiple sclerosis, Pemphigus, Pernicious anemia, Psoriasis, Reactive arthritis, Rheumatoid arthritis, Sjögren syndrome, Systemic lupus erythematosus, Type I diabetes, or Autoimmune hepatitis.

In an exemplary embodiment, the disclosure provides for a nucleic acid encoding a NT-3 polypeptide or a composition comprising said nucleic acid for use in the treatment of multiple sclerosis in a human subject in need thereof, wherein the viral vector comprises a nucleic acid encoding a NT-3 polypeptide; wherein a) the nucleic acid comprises a nucleotide sequence that is at least 90% identical to the nucleotide sequence of SEQ ID NO: 1; b) the nucleic acid comprises the nucleotide sequence of SEQ ID NO: 1; c) the nucleic acid comprises a nucleotide sequence encoding an amino acid sequence that is at least 90% identical to SEQ ID NO: 2; or d) the nucleic acid comprises a nucleotide sequence encoding the amino acid sequence of SEQ ID NO: 2.

The disclosure also provides for use of a nucleic acid encoding a NT-3 polypeptide for the preparation of a medicament for treating an autoimmune disease in a human subject in need thereof, wherein the medicament comprises a nucleic acid encoding a NT-3 polypeptide; wherein a) the nucleic acid comprises a nucleotide sequence that is at least 90% identical to the nucleotide sequence of SEQ ID NO: 1; b) the nucleic acid comprises the nucleotide sequence of SEQ ID NO: 1; c) the nucleic acid comprises a nucleotide sequence encoding an amino acid sequence that is at least 90% identical to SEQ ID NO: 2; or d) the nucleic acid comprises a nucleotide sequence encoding the amino acid sequence of SEQ ID NO: 2. For example, any of the disclosed medicaments are useful for treating autoimmune disease such as Alopecia areata, Addison disease, Celiac disease, Crohn's disease, Ulcerative colitis, Autoimmune inflammatory myositis, Graves disease, Hashimoto thyroiditis, inflammatory bowel disease, Multiple sclerosis, Pemphigus, Pernicious anemia, Psoriasis, Reactive arthritis, Rheumatoid arthritis, Sjögren syndrome, Systemic lupus erythematosus, Type I diabetes, or Autoimmune hepatitis.

In addition, the disclosure provides for use of a nucleic acid encoding a NT-3 polypeptide for the preparation of a medicament for treating multiple sclerosis in need thereof, wherein the medicament comprises a nucleic acid encoding a NT-3 polypeptide; wherein a) the nucleic acid comprises a nucleotide sequence that is at least 90% identical to the nucleotide sequence of SEQ ID NO: 1; b) the nucleic acid comprises the nucleotide sequence of SEQ ID NO: 1; c) the nucleic acid comprises a nucleotide sequence encoding an amino acid sequence that is at least 90% identical to SEQ ID NO: 2; or d) the nucleic acid comprises a nucleotide sequence encoding the amino acid sequence of SEQ ID NO: 2.

In any of the methods, uses or nucleic acids or compositions disclosed herein, a viral vector, such as a recombinant adeno-associated viral vector comprises the nucleic acid. In various embodiments, the viral vector is a recombinant adeno-associated virus (rAAV) vector. In related embodiments, the rAAV further comprises a pharmaceutically acceptable carrier. In various embodiments, the rAAV capsid serotype is AAV-1, AAV-2, AAV-3, AAV-4, AAV-5, AAV-6, AAV-7, AAV-8, AAV-9, AAV-10, AAV-11, AAV12, AAV13, Anc80, AAV-B1, AAVrh. 10, or AAVrh.74. In certain embodiments, the AAV capsid serotype is AAV-1.

Any of the methods, viral vectors or compositions of the disclosure can be carried out with a nucleic acid that is operatively linked to a muscle-specific transcriptional control element. The term “muscle specific control element” refers to a nucleotide sequence that regulates expression of a coding sequence that is specific for expression in muscle tissue. These control elements include enhancers and promoters. The disclosure provides for constructs comprising the muscle specific control elements MHCK7 promoter (Muscle Creatine Kinase promoter (7) w Hybrid intron), the MCK promoter and the MCK enhancer/or alpha myosin heavy chain (MHC) complex enhancer. In various embodiments, the muscle creatine kinase promoter sequence set out in SEQ ID NO: 11.

Exemplary muscle-specific promoter include one or more of a human skeletal actin gene element, a cardiac actin gene element, a desmin promoter, a skeletal alpha-actin (ASKA) promoter, a troponin I (TNNI2) promoter, a myocyte-specific enhancer binding factor MEF binding element, a muscle creatine kinase (MCK) promoter, a truncated MCK (tMCK) promoter, a myosin heavy chain (MHC) promoter, a hybrid a-myosin heavy chain enhancer-/MHC enhancer-promoter (MHCK7) promoter, a C5-12 promoter, a murine creatine kinase enhancer element, a skeletal fast-twitch troponin C gene element, a slow-twitch cardiac troponin c gene element, a slow-twitch troponin I gene element, hypoxia-inducible nuclear factor (HIF)-response element (HRE), a steroid-inducible element, and a glucocorticoid response element (GRE).

Any of the methods, compositions for use or uses of the disclosure can be carried out with a nucleic acid sequence that is the rAAV genome sequence comprising in order from 5′ to 3′: (i) a first AAV2 inverted terminal repeat sequence (ITR); (ii) a muscle creatine kinase promoter/enhancer sequence set out in nucleotides 147-860 of SEQ ID NO: 3; (iii) a nucleotide sequence encoding a human NT-3 polypeptide; and (iv) a second AAV2 ITR sequence; wherein the human NT-3 polypeptide has an amino acid sequence that is at least 90% identical to SEQ ID NO: 2 or is 100% identical to SEQ ID NO: 2, or is encoded by a nucleotide sequence at least 90% identical to nucleotides 1077-1850 of SEQ ID NO: 3 or 100% identical to nucleotides 1077-1850 of SEQ ID NO: 3.

Any of the methods, compositions for use or uses of the disclosure can be carried out with a nucleic acid which further comprising 3′ to the promoter/enhancer, a chimeric intron set out in nucleotides 892-1024 of SEQ ID NO: 3. In addition, the nucleic acids of the disclosure can further comprise 3′ to said nucleotide sequence encoding a human NT-3 polypeptide, a SV40 polyadenylation signal set out in nucleotides 1860-2059 of SEQ ID NO: 3.

In any of the methods, compositions for use or uses of the disclosure, the nucleic acids of the disclosure can comprise one or more inverted terminal repeat (ITR) sequences. For example, the nucleic can comprise a first ITR which is set out in nucleotides 7-112 of SEQ ID NO: 3, and/or a second ITR which is set out in nucleotides 2121-2248 of SEQ ID NO: 3.

In some embodiments, the nucleic acids comprise an scAAV1.tMCK.NT-3 genome that is at least 90% identical to SEQ ID NO: 9. In related embodiments, the nucleic acid comprising the scAAV1.tMCK.NT-3 genome is set out in SEQ ID NO: 9.

The disclosure provides for methods of treating an autoimmune disease, such as multiple sclerosis, in a human subject in need thereof comprising the step of administering to the human subject a dose of recombinant adeno-associated virus (rAAV) scAAV1.tMCK.NTF3 that results in sustained expression of a low concentration of NT-3 protein.

In any of the methods, compositions for use or uses of the disclosure, the nucleic acid or rAAV disclosed herein is administered at a dose that results in sustained expression of a low concentration of NT-3 polypeptide. In various embodiments, administering the nucleic acid or rAAV disclosed herein reduces inflammation in an organ affected by an autoimmune disease, such as MS, in the subject. In various embodiments, administering the nucleic acid or rAAV disclosed herein modulates an immune response in the subject. In various embodiments, administering the nucleic acid or rAAV disclosed herein increases the percentage of regulatory T cells in an organ affected by an autoimmune disease, such as MS, in the subject. In various embodiments, administering the nucleic acid or rAAV disclosed herein modulates the cytokine expression in dendritic cells in the subject.

In various embodiments, the nucleic acid viral vectors, rAAV, compositions or medicaments disclosed herein are administered intramuscularly. In related embodiments, the nucleic acid or rAAV disclosed herein is administered by intramuscular injection.

In any of the methods, compositions for use or uses of the disclosure, the subject is an older adult subject. In some embodiments, the subject is a pediatric subject, such as a subject less than 18 years old. In some embodiments, the subject is an adult (18 years or older). In particular, the subject is a young adult (18-39 years of age), middle aged adult (40-64 year of age) or an older adult or elderly subject (over 65 years of age) or a geriatric subject (over 70 years of age). In some embodiments, MS usually begins in an adult between the ages of 20 and 50. In some embodiments, MS is usually twice as common in women as in men.

### BRIEF DESCRIPTION OF THE DRAWINGS

The present disclosure may be more readily understood by reference to the following figures, wherein:

FIG. 1 provides a schematic of the cassette portion of the construct AAV.tMCK.NTF3 (set out in nucleotides 7-2248 of SEQ ID NO: 3; referred to herein as SEQ ID NO: 9). The rAAV contains the muscle specific tMCK promoter (SEQ ID NO: 11), chimeric intron (SEQ ID NO: 5), consensus Kozak sequence (SEQ ID NO: 6), the NTF3 cDNA (SEQ ID No: 1), and a polyadenylation signal (SEQ ID NO: 7).

FIG. 2 provides a restriction map and ORF Analysis of a cassette plasmid self-complementary pAAV.tMCK.NTF3 (SEQ ID NO: 3).

FIG. 3 provides the nucleotide sequence of a cassette production plasmid, self-complementary pAAV.tMCK.NTF3 (SEQ ID NO: 3).

FIG. 4 demonstrates AAV1.tMCK.NT-3 gene therapy induces detectable levels of NT-3 in serum. EAE mice were injected with a total of 1×10¹¹ vg of AAV1.tMCK.NT-3 into right gastrocnemius muscle. Blood samples were obtained from mice at 7 weeks post-injection, and NT-3 serum levels were determined by enzyme-linked immunosorbent assay. Data presents Mean±SEM (Female cohort: n=3 for untreated, and n=4 for NT-3 treated cohort; Male cohort: n=3 for both treated and untreated). * p<0.05.

FIG. 5 shows that treatment of NT-3 ameliorated the severity of experimental autoimmune encephalomyelitis (EAE) mouse model compared to untreated ones. The higher the clinical score indicates the worse physiological status of the mice. The overall score of NT-3 treated cohort is lower than untreated cohort. Data represents Mean±SEM (N=4 for untreated cohort and N=5 for NT-3 treated cohort). * p<0.05.

FIGS. 6A-6F provide the clinical scores of EAE mice (a, b) with or without AAV1.NT-3 treatment. Higher clinical scores indicate more severe symptoms in the mice. Red indicates the timepoint for AAV1.NTF3 injection. Grip strength (c, d) and rotarod (e, f) analyses for EAE mice with or without AAV1.NT-3 treatment. Data represents Mean±SEM (Female cohort: n=7 for untreated, and n=9 for NT-3 treated; male cohort: n=5 for untreated, and n=5 for NT-3 treated cohorts). * p<0.05.

FIG. 7 shows the results of behavior tests of NT-3 treated and untreated experimental autoimmune encephalomyelitis (EAE) mice. FIG. 7A shows results from rotarod assay; FIG. 7B shows results from grip strength assay. Data represents Mean±SEM (N=4 for untreated cohort; N=5 for NT-3 treated cohort). * p<0.05.

FIGS. 8A-8G provide Hematoxylin & Eosin (H&E) stained representative cross sections from lumbar spinal cord of (a) an untreated EAE mouse showing multifocal meningeal lymphocytic inflammation and (b) from AAV.NT-3 injected cohort (arrows depict inflammation). Luxor fast blue stained paraffin sections of lumbar cord from (c) untreated and (d) NT-3 treated EAE, and (e) from wildtype mice showing the presence of subpial demyelination (asterisks) in the untreated EAE mouse. Semi thick plastic sections from (f) an untreated mouse showing subpial axon loss (asterisk) contrasting with (g) a sample from NT-3 treated cohort, revealing preservation of the white matter long tracts in the descending anterolateral corticospinal tract at 7 weeks post treatment. Scale bar is 100 μm for a-e, and 50 μm for f and g.

FIGS. 9A-9H provides representative immunofluorescence images of anterior corticospinal tracks from (a) untreated (UT), (b) NT-3 treated (NT-3), and (c) wild type (WT) mice. (d) Bar graph shows the number of anti-neurofilament antibody positive particles representing axons counted in a predetermined area of the white matter in the anterior corticospinal tracks (UT, 52450.4±4851.3; NT-3, 71733.1±1778.0 axons/mm²; p=0.0203; n=3 for each cohort). Scale bar: 10 μm. The expression levels of (e, f) myelin basic protein (MBP) and (g, h) proteolipid protein (PLP) in the spinal cord from UT and NT-3 treated cohorts. MBP and PLP expression increased significantly with treatment in females (e, g).

FIG. 10 shows that NT-3 significantly reduced inflammation markers in disease affected organs in female mice. TNFα (FIG. 10A), IL6 (FIG. 10B), and IL1β (FIG. 10C) all reduced in NT-3 treated cohort in both brain and spinal cord. Data represents Mean±SEM of mice in each group (N=2 for untreated cohort; N=3 for treated cohort).

FIG. 11 provides expression levels of inflammatory markers TNFα, IL1β and IL6 in brain and spinal cord from EAE mice, (a-c) females and (d-f) males. TNFα, IL1B and IL6 all remarkably reduced in the NT-3 treated cohort (NT-3) in both brain and spinal cord compared to untreated (UT). In males IL1B expression is significant reduced in brain, and IL6 in spinal cord (e, f). Data represents Mean±SEM of mice in each group (female cohort: n=4 for untreated, n=4 for treated cohort; male cohort: n=3 for untreated, n=3 for treated cohort).

FIG. 12 shows that NT-3 significantly increased T regulatory cells (Treg cells) percentage in experimental autoimmune encephalomyelitis (EAE) mice. FIG. 12A shows cells from lymph node; FIG. 12B shows cells from spleen. Data represents Mean of mice in each group (N=2 for each cohort).

FIG. 13A-13C show T regulatory cells (Treg cells) percentage in lymph node and spleen from untreated and NT-3 treated EAE mice. (a) Representative figures of flow cytometry staining CD3+CD4+CD25+Foxp3+Tregs gated from CD3+CD4+ cell population. Population of Tregs in spleen and lymph node from (b) females and (c) from males. Data represents Mean±SEM (female cohort: n=3 for untreated, and n=4 for NT-3 treated cohort; male cohort: n=3 for untreated, and n=3 for treated cohort). * p<0.05

FIG. 14 shows that NT-3 significantly decreased TNFα expression in dendritic cells (DCs) challenged by myobacterium. DCs extracted from NT-3 treated and untreated experimental autoimmune encephalomyelitis (EAE) mice were isolated from bone marrow. Cultured DCs were challenged with mycobacterium for 24 hours, and the level of TNFα was determined as a marker for inflammation. Data represents Mean±SEM (N=4 for untreated cohort; N=5 for NT-3 treated cohort with female and male mice combined). * p<0.05.

FIG. 15A-15C demonstrates the tolerogenic feature of dendritic cells from bone marrow (a) Dendritic cells isolated from bone marrow of female EAE mice. Flow cytometry analysis showed 74.1% of total cultured cells expressed DC marker CD11c. DCs from NT-3 treated and untreated EAE (b) female and (c) male mice were challenged with mycobacterium and TNFα level was measured by qPCR. Data represents Mean±SEM (female cohort: n=4 for untreated, and n=4 for NT-3 treated cohort; male cohort: n=3 for untreated, and n=3 for NT-3 treated cohort). * p<0.05.

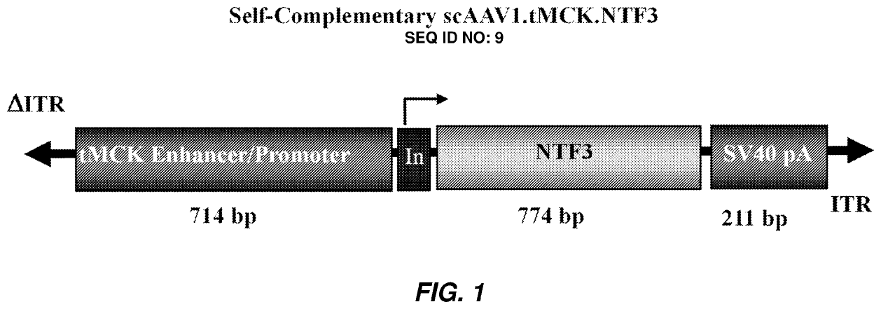

### DETAILED DESCRIPTION

The present disclosure relates to methods of treating autoimmune disease such as multiple sclerosis (MS). The methods disclosed herein comprise administering a therapeutically effective amount of neurotrophin-3 (NT-3), pro-NT-3, or an effective fragment thereof, or a nucleic acid encoding NT-3, pro-NT-3, or an effective fragment thereof, to a subject with multiple sclerosis.

NT-3 is an important growth factor stimulating glial cell survival and differentiation, and stimulates axon growth and myelination. NT-3 is also known to have immunomodulatory and anti-inflammatory properties as previously demonstrated in the spontaneous autoimmune peripheral polyneuropathy (SAPP) mouse model for chronic inflammatory polyradiculopathy (CIDP) in humans.

In one embodiment, the disclosure provides for gene therapy methods of treating autoimmune disease such as MS wherein the NT-3 encoding sequence of the NTF3 gene is delivered to the subject using self-complementary adeno-associated virus (scAAV) type 1 under control of a muscle-specific tMCK promoter.

Pre-clinical studies (see, Sahenk, Zarife, et al. “AAV1. NT-3 gene therapy for Charcot-Marie-Tooth neuropathy.” Molecular Therapy 22.3 (2014): 511-521) demonstrated that delivery of the construct AAV1.tMCK.NTF3 to the gastrocnemius muscle of the trembler J mice (Tr), a naturally occurring mouse model for CMT1, improved nerve regeneration, myelination, myelinated fiber density, sciatic nerve compound muscle action potential amplitude and functional performance on rotarod testing and hindlimb grip strength (see Example 1 of PCT/US2021/027279).

A major therapeutic goal in MS has been to block destructive immune effector cells while enhancing immunosuppressive regulatory cells. In the studies described herein, a gene therapy approach was administered to the EAE mouse model of MS via IM delivery of the AAV1.tMCK.NT-3 vector. The data provided herein demonstrated a systemic effect following transduction of muscle to produce NT-3 protein, which is released into serum, as detected by ELISA. This approach attenuated the clinical severity, provided histopathological evidence of remyelination supported by increased myelin protein gene expression and axon protection. The data herein also demonstrated that functional and histopathological improvements occurred in conjunction with an anti-inflammatory and immunomodulatory environment. Six to seven weeks following AAV1.NT-3 treatment, there was an increase of Treg cells population in both peripheral lymphocytes and splenocytes from both female and male cohorts. The increased Treg population was associated with suppressed inflammatory status in both spinal cord and brain as the inflammation markers TNFα, IL1B and IL6 were also significantly decreased or showed a trend in decreasing in treated cohorts. In MS patients, although Th1 and Th17 cells along with their associated cytokines IL-1, IL-6, IL-17, IFNγ and TNFα are increased, no significant difference in the frequency of Tregs were reported comparing to healthy controls (Danikowski et al., J Neuroinflammation; 14:117, 2017). However, Tregs from these patients are reported to have lower suppressive capabilities (Goswami et al., Hum Vaccin Immunother; 18:2035117, 2022), suggesting that that functional deficits in Tregs may contribute to the pathogenesis of MS. In addition, studies suggest that Tregs might be restricted from migrating to neuroinflammatory sites or undergo apoptosis upon arrival (Danikowski et al., J Neuroinflammation; 14:117, 2017).

Conceptually, CNS-derived DCs could be linked to Treg-cell expansion, thereby contributing to the resolution of CNS inflammation. DCs can modulate the expansion and function of Treg cells during CNS inflammation. Yet, in MS patients, it has been reported that DCs display an altered phenotype and there are dysfunctional interactions between DCs and Treg cells leading to loss of suppression of effector T cells resulting in myelin destruction, neuronal damage and neuroinflammation (Attfield et al., Nat Rev Immunol.; 22:734-750, 2022). Therefore, regulating the tolerogenic tolerance of DCs may be critical in treating MS. The data provided herein demonstrates that NT-3 suppressed DC tolerogenic responses in in vitro assays by isolating DCs from bone marrow and then challenge them using mycobacterium. The TNFα level was significantly suppressed in NT-3 treated DCs compared to untreated ones. Collectively, this data demonstrated the immunomodulatory role of NT-3 to suppress the autoimmune responses in the EAE mice.

### Multiple Sclerosis

Multiple sclerosis (MS) is the most common demyelinating disease that affects more than 2.5 million patients worldwide. MS, also known as encephalomyelitis disseminate, is an autoimmune disease of which the patients' immune system attacks the myelin of the nerves in brain and spinal cord, and eventually causes demyelination of the nerves leading to secondary axonal loss. In aspects, the present disclosure describes the use of AAV1.NT-3 gene therapy for treating multiple sclerosis.

In embodiments, the type of multiple sclerosis is relapsing, primary progressive, or secondary progressive.

In embodiments, the multiple sclerosis is a chronic progressive multiple sclerosis.

As used herein, the terms “treatment”, “treating”, and the like, refer to obtaining a desired pharmacologic or physiologic effect. The effect may be therapeutic in terms of a partial or complete cure for a disease or partial or complete inhibition or prevention of an adverse effect attributable to the disease. The terms “treatment”, “treating”, and the like may refer to improving clinical symptoms, slowing or preventing more adverse clinical symptoms, reducing or preventing an increase in inflammation, and/or modulating the immune response. “Treatment”, as used herein, covers any treatment of a disease in a mammal, particularly in a human, and can include inhibiting the disease or condition, i.e., arresting its development; and relieving the disease, i.e., causing regression of the disease.

“Prevention”, as used herein, refers to any action providing a benefit to a subject at risk of being afflicted with a condition or autoimmune disease such as multiple sclerosis.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of an agent which will achieve the goal of improvement in disease severity and the frequency of incidence. The effectiveness of treatment may be measured by evaluating a reduction in symptoms in a subject in response to the administration of NT-3.

The term “effective fragment” refers to a portion of the polynucleotide sequence encoding a functional fragment of the NT-3 polypeptide. The term “effective fragment” also refers to a portion of the NT-3 polypeptide amino acid sequence that retains NT-3 growth factor activity. Exemplary NT-3 growth factor activities include supporting the survival and differentiation of existing neurons, and inducing and supporting the growth and differentiation of new neurons and synapses. In addition, NT-3 activity includes stimulating muscle growth and muscle function, activating the mTOR signaling, increasing muscle fiber diameter, and/or increasing muscle contractility. In other aspects, NT-3 activity includes reducing inflammation in an organ affected by MS or an autoimmune disease in the subject, wherein the organ is a brain, a spinal cord, joints, muscles, skin, pancreas, liver, or kidney; reducing inflammation; modulating an immune response; increasing the percentage of regulatory T cells in an organ affected by MS or an autoimmune disease, wherein the organ is a lymph node, spleen, thymus, or peripheral blood; or modulating the cytokine expression in dendritic cells.

As used herein, the term “diagnosis” can encompass determining the likelihood that a subject will develop a disease (e.g., without limitations, an MS or another autoimmune disease), or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., without limitations, adjustment of dose or dosage regimen), and the like.

A “subject,” as used herein, can be any animal, and may also be referred to as the patient. Preferably the subject is a vertebrate animal, and more preferably the subject is a mammal, such as a domesticated farm animal (e.g., cow, horse, pig, sheep) or pet (e.g., dog, cat, guinea pig). In some embodiments, the subject is a human. In some embodiments, the subject is an adult (18 years or older). In particular, the subject is a young adult (18-39 years of age), middle aged adult (40-64 year of age) or an older adult or elderly subject (over 65 years of age) or a geriatric subject (over 70 years of age). In some embodiments, MS usually begins in an adult between the ages of 20 and 50.

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native inter-nucleoside bonds, or both. The nucleic acid can be in any topological conformation. For instance, the nucleic acid can be single-stranded, double-stranded, triple-stranded, quadruplexed, partially double-stranded, branched, hair-pinned, circular, or in a padlocked conformation.

The term “gene” as used herein refers to a nucleotide sequence that can direct synthesis of an enzyme or other polypeptide molecule (e.g., can comprise coding sequences, for example, a contiguous open reading frame (ORF) which encodes a polypeptide) or can itself be functional in the organism. A gene in an organism can be clustered within an operon, as defined herein, wherein the operon is separated from other genes and/or operons by intergenic DNA. Individual genes contained within an operon can overlap without intergenic DNA between the individual genes.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

The term “vector” or “expression vector” refers to any type of genetic construct comprising a nucleic acid coding for an RNA capable of being transcribed. Expression vectors can contain a variety of control sequences, structural genes (e.g., genes of interest), and nucleic acid sequences that serve other functions as well.

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

A “recombinant AAV (rAAV)” as used herein refers to a viral vector comprising one or more polynucleotides of interest (or transgenes) that are flanked by AAV terminal repeat sequences (ITRs). Such rAAV can be replicated and packaged into infectious viral particles when present in a host cell that has been transfected with a vector encoding and expressing rep and cap gene products.

A “rAAV virion” or “rAAV viral particle” or “rAAV vector particle” refers to a viral particle composed of at least one AAV capsid protein and an encapsidated polynucleotide rAAV. If the particle comprises a heterologous polynucleotide (i.e., a polynucleotide other than a wild-type AAV genome such as a transgene to be delivered to a mammalian cell), it is typically referred to as a “rAAV vector particle” or simply “rAAV particle.” Thus, production of rAAV vector particle necessarily includes production of rAAV, as such a rAAV genome is contained within an rAAV vector particle.

As used herein, the term “about” refers to +/−10% deviation from the basic value.

Unless defined otherwise, all technical and scientific terms used herein have the same meaning as commonly understood by one of ordinary skill in the art to which this disclosure belongs.

### Autoimmune Disease

In one aspect, the present disclosure provides methods of treating a subject having autoimmune disease using gene therapy of the present disclosure. An “autoimmune disease” refers to a condition, disease or disorder wherein the subject's immune response targets its own functioning cells, tissues, and/or organs.

In embodiments, some common diseases that are generally considered autoimmune include celiac disease, diabetes mellitus type 1, graves' disease, inflammatory bowel disease, multiple sclerosis, alopecia areata, Addison's disease, pernicious anemia, psoriasis, rheumatoid arthritis, and systemic lupus erythematosus. The presence and severity of symptoms of an autoimmune disease will depend on the location and type of autoimmune response that occurs. A person may have more than one autoimmune disease simultaneously and display symptoms of each. Signs and symptoms presented, and the disease itself, can depend on age, hormones, environment, and other factors. In general, the common symptoms are fatigue, low fever, generally feeling unwell (malaise), muscle aches and joint pain, and rash.

Since multiple sclerosis is also a type of autoimmune disease, certain information provided in the previous section (i.e., under “Multiple Sclerosis”) are also applicable to this current section (i.e., under “Autoimmune Disease”).

In other aspects, the autoimmune disease is Alopecia areata, Addison disease, Celiac disease, Crohn's disease, Ulcerative colitis, Autoimmune inflammatory myositis, Graves disease, Hashimoto thyroiditis, inflammatory bowel disease, Multiple sclerosis, Pemphigus, Pernicious anemia, Psoriasis, Reactive arthritis, Rheumatoid arthritis, Sjögren syndrome, Systemic lupus erythematosus, Type I diabetes, or Autoimmune hepatitis.

As used herein, the terms “treatment”, “treating”, and the like, when using in reference to autoimmune disease refer to obtaining a desired pharmacologic or physiologic effect. The effect may be therapeutic in terms of a partial or complete cure for a disease or partial or complete inhibition or prevention of an adverse effect attributable to the autoimmune disease. The terms “treatment”, “treating”, and the like may also refer to improving clinical symptoms, slowing or preventing more adverse clinical symptoms, reducing the severity or preventing the symptoms or prevalence of autoimmune disease, or modulating, reducing or preventing the autoimmune response or inflammation.

“Treatment”, as used herein, covers any treatment of a disease in a mammal, particularly in a human, and can include inhibiting the disease or condition, i.e., arresting its development; and relieving the disease, i.e., causing regression of the disease or causing regression of the autoimmune response or inflammation.

Gene Therapy for Multiple Sclerosis and/or Autoimmune Disease

In one aspect, the present disclosure provides methods of treating a subject having an autoimmune disease, such as multiple sclerosis, using gene therapy.

In embodiments, the method, the viral vector, composition for use or the use of the present disclosure is carried out in combination with one or more other therapy (e.g., without limitations, a therapy commonly used to treat multiple sclerosis and/or autoimmune disease).

Vectors which can be used to deliver a therapeutic nucleic acid include viral and non-viral vectors. Suitable vectors which can be used include adenovirus, adeno-associated virus, retrovirus, lentivirus, HSV (herpes simplex virus) and plasmids. An advantage of Herpes simplex virus vectors is their natural tropism for sensory neurons. However, adenovirus associated viral vectors are most popular, due to their low risk of insertional mutagenesis and immunogenicity, their lack of endogenous viral genes, and their ability to be produced at high titer. Kantor et al. review a variety of methods of gene transfer to the central nervous system, while Goins et al. describe methods of gene therapy for the treatment of chronic peripheral nervous system pain. See Kantor et al., Adv Genet. 87, 125-197 (2014), and Goins et al., Neurobiol. Dis. 48 (2), 255-270 (2012), the disclosures of which are incorporated herein by reference. In particular, successful gene delivery to Schwann cells, the resident glia cells of peripheral nerves, has been reported using various viral vectors. Mason et al., Curr. Gene Ther.11, 75-89 (2011). If the vector is in a viral vector and the vector has been packaged, then the virions can be used to infect cells. If naked DNA is used, then transfection or transformation procedures as are appropriate for the particular host cells can be used. Formulations of naked DNA utilizing polymers, liposomes, or nanospheres can be used for gene delivery. Nucleic acids can be administered in any desired format that provides sufficiently efficient delivery levels, including in virus particles, in liposomes, in nanoparticles, and complexed to polymers.

The nucleic acid (e.g., cDNA or transgene) encoding a gene whose expression treats (e.g., without limitations, decreases or prevents) an autoimmune disease, such as multiple sclerosis can be cloned into an expression cassette that has a regulatory element such as a promoter (constitutive or regulatable) to drive transgene expression and a polyadenylation sequence downstream of the nucleic acid. For example, regulatory elements that are 1) specific to a tissue or region of the body; 2) constitutive; and/or 3) inducible/regulatable can be used.

In some embodiments, muscle-specific transcriptional regulatory elements are used. Muscle-specific regulatory elements include muscle-specific promoters including mammalian muscle creatine kinase (MCK) promoter, a truncated MCK (tMCK) promoter, a myosin heavy chain (MHC) promoter, a hybrid a-myosin heavy chain enhancer-/MHC enhancer-promoter (MHCK7) promoter, a C5-12 promoter, mammalian desmin promoter, mammalian troponin I (TNNI2) promoter, or mammalian skeletal alpha-actin (ASKA) promoter. The development and improvement of muscle-specific promoters are generally based on skeletal muscle α-actin, muscle creatine kinase, and desmin genes, as well as other genes expressed in muscles. Muscle-specific enhancers useful in the present disclosure are selected from the group consisting of mammalian MCK enhancer, mammalian DES enhancer, and vertebrate troponin I IRE (TNI IRE, herein after referred to as FIRE) enhancer, a skeletal fast-twitch troponin C gene element, a slow-twitch cardiac troponin c gene element, a slow-twitch troponin I gene element, hypoxia-inducible nuclear factor (HIF)-response element (HRE), a steroid-inducible element, and a glucocorticoid response element (GRE). One or more of these muscle-specific enhancer elements may be used in combination with a muscle-specific promoter of the disclosure to provide a tissue-specific regulatory element.

A preferred viral vector for use in treating multiple sclerosis or autoimmune disease by gene therapy is AAV. AAV-mediated gene delivery has emerged as an effective and safe tool for both preclinical and clinical studies of neurological disorders. Ojala et al., Neuroscientist., 21 (1): 84-98 (2015). Currently, rAAV is the most widely used vector for clinical trials for neurological disorders, and no adverse effects linked to the use of this vector have ever been reported from clinical trials: Adeno-associated virus (AAV) is a non-pathogenic dependovirus from the parvoviridae family requiring helper functions from other viruses, such as adenovirus or herpes simplex virus, to fulfill its life cycle. The wild-type (WT) AAV is characterized by a single-stranded DNA (ssDNA) genome, with inverted terminal repeats (ITR) at both ends, of approximately 5 kb surrounded by a capsid.

Adenoviral vectors for use to deliver transgenes to cells for applications such as in vivo gene therapy and in vitro study and/or production of the products of transgenes, commonly are derived from adenoviruses by deletion of the early region 1 (El) genes (Berkner, K. L., Curr. Top. Micro. Immunol. 158 L39-66 1992). Deletion of El genes renders such adenoviral vectors replication defective and significantly reduces expression of the remaining viral genes present within the vector. Recombinant adenoviral vectors have several advantages for use as gene delivery vehicles, including tropism for both dividing and non-dividing cells, minimal pathogenic potential, ability to replicate to high titer for preparation of vector stocks, and the potential to carry large inserts. However, it is believed that the presence of the remaining viral genes in adenoviral vectors can be deleterious.

Accordingly, in some embodiments, adenoviral vectors with deletions of various adenoviral gene sequences. In particular, pseudoadenoviral vectors (PAVs), also known as ‘gutless adenovirus’ or mini-adenoviral vectors, are adenoviral vectors derived from the genome of an adenovirus that contain minimal cis-acting nucleotide sequences required for the replication and packaging of the vector genome and which can contain one or more transgenes (See, U.S. Pat. No. 5,882,877 which covers pseudoadenoviral vectors (PAV) and methods for producing PAV, incorporated herein by reference). Such PAVs, which can accommodate up to about 36 kb of foreign nucleic acid, are advantageous because the carrying capacity of the vector is optimized, while the potential for host immune responses to the vector or the generation of replication-competent viruses is reduced. PAV vectors contain the 5′ inverted terminal repeat (ITR) and the 3′ ITR nucleotide sequences that contain the origin of replication, and the cis acting nucleotide sequence required for packaging of the PAV genome, and can accommodate one or more transgenes with appropriate regulatory elements, e.g., promoter, enhancers, etc.

### AAV

Recombinant AAV (rAAV) genomes of the disclosure comprise nucleic acid molecule of the disclosure and one or more AAV ITRs flanking a nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, Anc80, AAVrh.74, AAVrh. 10, and AAV-B1 (see, e.g., Gao et al., PNAS, 99:11854-11859 (2002)) and any variants thereof; and Viral Vectors for Gene Therapy: Methods and Protocols, ed. Machida, Humana Press, 2003). Furthermore, pseudotyped rAAV vectors may also be utilized in the methods described herein. Pseudotyped rAAV vectors are those which contain the genome of one AAV serotype in the capsid of a second AAV serotype; for example, an rAAV vector that contains the AAV2 capsid and the AAV1 genome or an rAAV vector that contains the AAV5 capsid and the AAV2 genome. (Auricchio et al., (2001) Hum. Mol. Genet., 10 (26): 3075-81). Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). As noted in the Background section above, the nucleotide sequences of the genomes of various AAV serotypes are known in the art. To promote skeletal muscle specific expression, AAV1, AAV6, AAV8, or AAVrh.74 may be used.

DNA plasmids of the disclosure comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an rAAV genome to be packaged, rep and cap genes, and helper virus functions are provided to a cell, are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV12, AAV13, Anc80, AAV-B1, AAVrh. 10, or AAVrh.74 and variants thereof. Production of pseudotyped rAAV is disclosed in, for example, WO 01/83692 which is incorporated by reference herein in its entirety.

A method of generating a packaging cell is to create a cell line that stably expresses all the necessary components for rAAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. rAAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as Hela cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells), and FRhL-2 cells (rhesus fetal lung cells).

Recombinant AAV particles (i.e., infectious encapsidated rAAV particles) of the disclosure comprise a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the disclosure are set out in International Patent Application No. PCT/US2012/047999 (WO 2013/016352) incorporated by reference herein in its entirety.

The rAAV may be purified by methods standard in the art such as by column chromatography or cesium chloride gradients. Methods for purifying rAAV from helper virus are known in the art and include methods disclosed in, for example, Clark et al., Hum. Gene Ther., 10 (6): 1031-1039 (1999); Schenpp and Clark, Methods Mol. Med., 69 427-443 (2002); U.S. Pat. No. 6,566,118 and WO 98/09657.

In another embodiment, the disclosure contemplates compositions comprising rAAV of the present disclosure. Compositions of the disclosure comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG).

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg).

Methods of transducing a target cell with rAAV, in vivo or in vitro, are contemplated by the disclosure. The in vivo methods comprise the step of administering an effective dose, or effective multiple doses, of a composition comprising an rAAV of the disclosure to an animal (including, without limitations, a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (e.g., without limitations, eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

In particular, actual administration of rAAV of the present disclosure may be accomplished by using any physical method that will transport the rAAV into the target tissue of an animal. Administration according to the disclosure includes, but is not limited to, injection into muscle, the bloodstream and/or directly into the liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The rAAV can be used with any pharmaceutically acceptable carrier for ease of administration and handling.

Transduction may be carried out with gene cassettes comprising tissue specific control elements. For example, one embodiment of the disclosure provides methods of transducing muscle cells and muscle tissues directed by muscle specific control elements, including, but not limited to, those derived from the actin and myosin gene families, such as from the myoD gene family [See Weintraub et al., Science, 251:761-766 (1991)], the myocyte-specific enhancer binding factor MEF-2 (Cserjesi and Olson, Mol Cell Biol 11:4854-4862 (1991)), control elements derived from the human skeletal actin gene (Muscat et al., Mol Cell Biol, 7:4089-4099 (1987)), the cardiac actin gene, muscle creatine kinase sequence elements (See Johnson et al., Mol Cell Biol, 9:3393-3399 (1989)) and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors (Semenza et al., Proc Natl Acad Sci USA, 88:5680-5684 (1991)), steroid-inducible elements and promoters including the glucocorticoid response element (GRE) (See Mader and White, Proc. Natl. Acad. Sci. USA 90:5603-5607 (1993)), and other control elements.

Muscle tissue is an attractive target for in vivo DNA delivery, because it is easy to access and gene expression can be confined to muscle with muscle specific promoter, and NT-3 is naturally produced by muscle. The disclosure contemplates sustained expression of NT-3 from transduced myofibers.

By “muscle cell” or “muscle tissue” is meant a cell or group of cells derived from muscle of any kind (for example, skeletal muscle and smooth muscle, e.g., from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “transduction” is used to refer to the administration/delivery of the coding region of NT-3 to a recipient cell either in vivo or in vitro, via a replication-deficient rAAV of the disclosure resulting in expression of NT-3 by the recipient cell.

In one embodiment, the gene therapy is NT-3 gene therapy via rAAV delivery. An AAV expression cassette carrying human NT-3 cDNA coding sequence under the control of the triple muscle-specific creatine kinase (tMCK) promoter is disclosed herein. It has been previously shown that an improvement in motor function, histopathology, and electrophysiology of peripheral nerves can be achieved using AAV1 to increase neurotrophin-3 expression in the tremble (Try) mouse, which is a model for the Charcot-Marie-Tooth disease variant CMT1A. See Sahenk et al., Mol Ther. 22 (3): 511-21 (2014), the disclosure of which is incorporated herein by reference.

Thus, the disclosure provides methods of administering an effective dose (or doses, administered essentially simultaneously or doses given at intervals) of rAAV that encode NT-3 to a patient in need thereof.

### Doses and Routes of Administration

The disclosure provides for local administration and systemic administration of an effective dose of rAAV and compositions of the disclosure including combination therapy of the disclosure. For example, systemic administration is administration into the circulatory system so that the entire body is affected. Systemic administration includes enteral administration such as absorption through the gastrointestinal tract and parental administration through injection, infusion, or implantations.

Routes of administration for the rAAV contemplated in the foregoing methods therefore include, but are not limited to, intraperitoneal (IP), intramuscular (IM) and intravascular including, for example, inter-arterial limb perfusion (ILP) and intravenous (IV) routes.

The dose of rAAV to be administered in methods disclosed herein will vary depending, for example, on the particular rAAV, the mode of administration, the treatment goal, the individual, and the cell type(s) being targeted, and may be determined by methods standard in the art. More than one dose may be administered, for example, one, two, three or more doses. Titers of rAAV in a dose may range from about 1×10⁶, about 1×10⁷, about 1×10⁸, about 1×10⁹, about 1×10¹⁰, about 1×111, about 1×10¹², about 1.5×10¹², about 1×10¹², about 3×10¹², about 4×10¹², about 5×10¹², about 6×10¹², about 6.5×10¹², about 7×10¹², 1×10¹³, about 1×10¹⁴, or to about 1×10¹⁵ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, about 1.5×10¹² vg, about 1×10¹² vg, about 3×10¹² vg, about 4×10¹² vg, about 5×10¹² vg, about 6×10¹² vg, about 6.5×10¹² vg, about 7×10¹² vg, 1×10¹³ vg, 1×10¹⁴ vg, 1×10¹⁵ respectively). Methods for titering rAAV are described in Clark et al., Hum. Gene Ther., 10:1031-1039 (1999). In embodiments, dosages may also be expressed in units of viral genomes (vg/kg) (i.e., 1×10⁷ vg/kg, 1×10⁸ vg/kg, 1×10⁹ vg/kg, 1×10¹⁰ vg/kg, 1×10¹¹ vg/kg, 1×10¹² vg/kg, about 1.5×10¹² vg/kg, about 1×10¹² vg/kg, about 3×10¹² vg/kg, about 4×10¹² vg/kg, about 5×10¹² vg/kg, about 6×10¹² vg/kg, about 6.5×10¹² vg/kg, about 7×10¹² vg/kg, 1×10¹³ vg/kg, 1×10¹⁴ vg/kg, 1×10¹⁵ vg/kg respectively).

In some embodiments of the foregoing methods in which the route of administration is an IM route, the dose of the rAAV administered is from about 1.5×10¹² to at least about 6.5×10¹² vg/kg. (All ranges herein are intended to represent each individual value in the ranges, as well as the individual upper and lower values of each range.) In some embodiments of the foregoing methods in which the route of administration is IM, the dose of the rAAV administered is 2×10¹² vg/kg. In some embodiments of the foregoing methods in which the route of administration is IM, the dose of the rAAV administered is 4×10¹² vg/kg. In some embodiments of the foregoing methods in which the route of administration is IM, the dose of the rAAV administered is 6×10¹² vg/kg.

Human patients are subjects contemplated herein for treatment. Human patients are subjects contemplated herein for treatment by IM delivery. Such patients include those patients that i) adult subjects (18 years or older) diagnosed with multiple sclerosis or autoimmune disease, and in particular older adults (>65 years), ii) males and females of any ethnic or racial group. In embodiments, the subject is a pediatric subject, such as a subject less than 18 years old. For example, some patients may be slowed down by aging and are experiencing difficulty to go up and down steps or getting up from sitting position, slowed walking, easy fatigue, difficulty with balance, difficulty raising arms above shoulders or keeping erect posture. Suitable patients may not include, e.g., those with i) active viral infection based on clinical observations or serological evidence of HIV, or Hepatitis A, B or C infection, ii) ongoing immunosuppressive therapy or immunosuppressive therapy within 6 months of starting the trial (e.g., corticosteroids, cyclosporine, tacrolimus, methotrexate, cyclophosphamide, intravenous immunoglobulin), iii) persistent leukopenia or leukocytosis (WBC≤3.5 K/μL or ≥20.0 K/μL) or an absolute neutrophil count <1.5K/μL, iv) AAV1 binding antibody titers ≥1:50 as determined by ELISA immunoassay, v) concomitant illness or requirement for chronic drug treatment that in the opinion of the PI creates unnecessary risks for gene transfer, vi) ankle contractures or surgeries preventing proper muscle strength testing, vii) pregnancy, breast feeding, or plans to become pregnant, viii) other causes of neuropathy, and/or ix) limb surgery in the past six months. In an exemplary clinical protocol, multiple sclerosis or autoimmune disease patients receive a total dose of scAAV1.tMCK.NTF3 divided into medial and lateral heads of the gastrocnemius and tibialis anterior (TA) muscles of legs which are preferentially exhibiting muscle weakness and instability. Subjects receive one of the following: i) low dose of rAAV of 2×10¹² vg/kg (total dose) or ii) a high dose of rAAV of 6×10¹² vg/kg (total dose).

In one embodiment, the rAAV is administered by IM injection without diluent. In alternative embodiments, compositions for intramuscular injection include an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

In another aspect, rAAV genomes are provided herein. The genomes of the rAAV administered comprise a NT-3 polynucleotide under the control of transcription control sequences. The rAAV genomes lack AAV rep and cap DNA. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV12, AAV13, Anc80, AAV-B1, AAVrh. 10, AAVrh.74, or variants thereof. The nucleotide sequences of the genomes of these AAV serotypes are known in the art as noted in the Background Section above.

In some embodiments, the transcription control sequences of the rAAV genomes are muscle-specific control elements, including, but not limited to, those derived from the actin and myosin gene families, such as from the myoD gene family [See Weintraub et al., Science, 251:761-766 (1991)], the myocyte-specific enhancer binding factor MEF-2 [Cserjesi and Olson, Mol. Cell. Biol., 11:4854-4862 (1991)], control elements derived from the human skeletal actin gene [Muscat et al., Mol. Cell. Biol., 7:4089-4099 (1987)], the cardiac actin gene, muscle creatine kinase (MCK) promoter [Johnson et al., Mol. Cell. Biol., 9:3393-3399 (1989)] and the MCK enhancer, MHCK7 promoter (a modified version of MCK promoter that incorporates an enhancer from myosin heavy chain (Salva et al., Mol. Ther., 15:320-329 (2007)), desmin promoter, control elements derived from the skeletal fast-twitch troponin C gene, the slow-twitch cardiac troponin C gene and the slow-twitch troponin I gene: hypoxia-inducible nuclear factors (Semenza et al., Proc. Natl. Acad. Sci. USA, 88:5680-5684 (1991)), steroid-inducible elements and promoters including the glucocorticoid response element (GRE) (See Mader and White, Proc. Natl. Acad. Sci. USA, 90:5603-5607 (1993)), and other control elements. In some embodiments, the transcription control elements include the MCK promoter/enhancer which is included in the AAV.tMCK.NTF3 genome disclosed herein. The MCK promoter/enhancer is composed of the muscle creatine kinase promoter with an added enhancer element (enh358MCK, 584-bp) fused to it. A triple tandem of the MCK enhancer (206-bp) was ligated to the 87-bp basal promoter in the tMCK promoter/enhancer. In some embodiments, the transcription control elements and the tMCK promoter/enhancer is included in the AAV.tMCK.NTF3 genome as set out in SEQ ID NO: 9. In some embodiments, the tMCK promoter/enhancer is according to the nucleotide sequence of SEQ ID NO: 11.

In some embodiments, the NT-3 polynucleotide in a rAAV genome is the NT-3 cDNA set out in SEQ ID NO: 1 (corresponding to nucleotides 1077-1850 of SEQ ID NO: 3). In some embodiments, the NT-3 polynucleotide in a rAAV genome is the NT-3 cDNA set out in GenBank Accession #NM_001102654 or the NTF3 cDNA sequence set out as SEQ ID NO: 1, or is a variant polynucleotide having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the NT-3 cDNA sequence set out as SEQ ID NO: 1. In some embodiments, the variant NT-3 polynucleotide encodes the same NT-3 polypeptide as the polypeptide encoded by the NT-3 cDNA of SEQ ID NO: 1. The amino acid sequence of the NT-3 polypeptide encoded by the NT-3 cDNA set out as SEQ ID NO: 1 or provided as GenBank Accession #NM_001102654 is set out in SEQ ID NO:2. In some embodiments, the variant NT-3 polynucleotide encodes a variant NT-3 polypeptide with at least one amino acid sequence alteration as compared to the amino acid sequence of the polypeptide (SEQ ID NO: 2) encoded by NT-3 cDNA set out in SEQ ID NO: 1 or provided as GenBank Accession #NM_001102654. An amino acid sequence alteration can be, for example, a substitution, a deletion, or an insertion of one or more amino acids, preferably conservative substitutions. A variant NT-3 polypeptide can have any combination of amino acid substitutions, deletions or insertions where activity of the polypeptide is retained. In one aspect, a variant NT-3 polypeptide can have a number of amino acid alterations such that its amino acid sequence shares at least 60%, 70%, 80%, 85%, 86%, 87%, 88%, 89%, 90%, 91%, 92%, 93%, 94%, 95%, 96%, 97%, 98%, 99%, or 99.5% identity with the amino acid sequence (SEQ ID NO: 2) encoded by NT-3 cDNA set out as SEQ ID NO: 1 or provided as GenBank Accession #NM_001102654.

In some embodiments, the rAAV genome is the AAV.tMCK.NTF3 genome, the sequence of the NT-3 gene cassette of which is set out in SEQ ID NO: 9.

In yet another aspect, an isolated nucleic acid comprising the nucleotide sequence set out in nucleotides 7-2248 of SEQ ID NO: 3 is provided. In some embodiments, the isolated nucleic acid consists of the nucleotide sequence set out in nucleotides 7-2248 of SEQ ID NO: 3.

Also provided is an isolated nucleic acid comprising, in order from 5′ to 3′: (i) a first AAV2 inverted terminal repeat sequence (ITR) (SEQ ID NO: 4); (ii) a muscle creatine kinase promoter sequence (set out in nucleotides 147-860 of SEQ ID NO: 3); (iii) a nucleotide sequence encoding a human NT-3 polypeptide (SEQ ID NO: 1); and (iv) a second AAV2 ITR sequence (SEQ ID NO: 8), wherein the human NT-3 polypeptide has an amino acid sequence that is at least 90% identical to SEQ ID NO: 2, is 100% identical to SEQ ID NO:2, or is encoded by nucleotides 1077-1850 of SEQ ID NO: 3.

Recombinant AAV comprising the foregoing nucleic acids are contemplated as well as rAAV comprising a nucleotide sequence that is at least 90% identical to the nucleotide sequence set out in SEQ ID NO: 1.

DNA plasmids comprising rAAV genomes of the disclosure are provided. The DNA plasmids comprise rAAV genomes contemplated herein. An exemplary DNA plasmid is provided as SEQ ID NO: 3 and annotated in Table 3 (see Example 2). The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV12, AAV13, Anc80, AAV-B1, AAVrh.10, AAVrh.74, or variants thereof. Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014).

A method of generating a packaging cell is to create a cell line that stably expresses all the necessary components for rAAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. rAAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells. Methods for producing rAAV with self-complementary genomes are also known in the art.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

In a further aspect, the disclosure thus provides packaging cells that produce infectious rAAV. In one embodiment packaging cells may be stably transformed cancer cells such as Hela cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells), and FRhL-2 cells (rhesus fetal lung cells).

The rAAV may be purified by methods standard in the art such as by column chromatography or cesium chloride gradients. Methods for purifying rAAV from helper virus are known in the art and include methods disclosed in, for example, Clark et al., Hum. Gene Ther., 10 (6): 1031-1039 (1999); Schenpp and Clark, Methods Mol. Med., 69 427-443 (2002); U.S. Pat. No. 6,566,118 and WO 98/09657.

Thus, in another aspect, the disclosure contemplates a rAAV comprising a NT-3 polynucleotide. In some embodiments, the rAAV comprises AAVrh74 capsid and a NT-3 polynucleotide. In some embodiments, the genome of the rAAV lacks AAV rep and cap DNA. In some embodiments of the methods, the rAAV is rAAVrh.74.tMCK.NTF3. In some embodiments, the rAAV is a self-complementary genome.

In another aspect, the disclosure contemplates compositions comprising a rAAV described herein. Compositions of the disclosure comprise rAAV in a pharmaceutically acceptable carrier. The compositions may also comprise other ingredients such as diluents. Acceptable carriers and diluents are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG). In some embodiments, the rAAV is formulated in Tris, MgCl₂, NaCl and pluronic F68. In some embodiments, the rAAV is formulated in 20 mM Tris (pH 8.0), 1 mM MgCl₂ and 200 mM NaCl containing 0.001% pluronic F68.

Combination treatments are also contemplated herein. Combinations as used herein include simultaneous treatment or sequential treatments. Combinations of methods of the disclosure with standard medical treatments (e.g., corticosteroids and/or immunosuppressive drugs) are specifically contemplated, as are combinations with novel treatments. In various embodiments, subjects are treated with corticosteroids before, during or after (or with any permutation of combinations of two or more of the three possibilities), the subject is treated according to a method contemplated herein. For example, the combinations include administering a corticosteroid, e.g., prednisolone, before, during and/or after administration of the rAAV.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

### Neurotophin-3

In some embodiments, a therapeutically effective amount of NT-3, pro-NT-3, or an NT-3 analog thereof is administered to the subject to stimulate muscle growth. Neurotrophin 3 (NT-3) is a neurotrophic factor in the NGF (Nerve Growth Factor) family of neurotrophins. NT-3 is a protein growth factor which has activity on certain neurons of the peripheral and central nervous system; it is best known for helping to support the survival and differentiation of existing neurons, and encourages the growth and differentiation of new neurons and synapses.

As used herein, the term “polypeptide” refers to an oligopeptide, peptide, or protein sequence, or to a fragment, portion, or subunit of any of these, and to naturally occurring or synthetic molecules. The term “polypeptide” also includes amino acids joined to each other by peptide bonds or modified peptide bonds, i.e., peptide isosteres, and may contain any type of modified amino acids, The term “polypeptide” also includes peptides and polypeptide fragments, motifs and the like, glycosylated polypeptides, all “mimetic” and “peptidomimctic” polypeptide forms, and retro-inversion peptides (also referred to as all-D-retro or mtro-enantio peptides).

“Substantially similar” means that a given amino acid (or nucleic acid) sequence shares at least 85%, 86%, 87%, 88%, 89%, 90%, 91%, 92%, 93%, 94%, 95%, 96%, 97%, 98%, or 99% identity with a reference sequence. In various embodiments, “substantially similar” means that a given amino acid (or nucleic acid) sequence shares at least 85%, more preferably at least 90%, and even more preferably at least 95% identity with a reference sequence. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. N-terminal, C-terminal or internal extensions, deletions, or insertions into the peptide sequence shall not be construed as affecting homology.

Substantially similar peptides include those that differ by one or more amino acid alterations, where the alterations, e.g., substitutions, additions or deletions of amino acid residues, do not abolish the properties of the relevant peptides, such as their ability to associate with FAK or NANOG. Furthermore, only sequences describing or encoding proteins in which only conservative substitutions are made in the conserved regions are substantially similar overall. Preferable, substantially similar sequences also retain the distinctive activity of the poly peptide.

Examples of conservative substitutions include the substitution of a non-polar (hydrophobic) residue such as isoleucine, leucine, or methionine for another. Likewise, the present disclosure contemplates the substitution of one polar (hydrophilic) residue such as between arginine and lysine, between glutamine and asparagine, and between glycine and serine. Additionally, the substitution of a basic residue such as lysine, arginine or histidine for another or the substitution of one acidic residue such as aspartic acid or glutamic acid for another is also contemplated. Examples of non-conservative substitutions include the substitution of a non-polar (hydrophobic) residue such as isoleucine, valine, leucine, alanine, methionine for a polar (hydrophilic) residues such as cysteine, glutamine, glutamic acid, lysine and/or a polar residue for a non-polar residue.

The phrase “conservative substitution” also includes the use of chemically derivatized residues in place of a non-derivatized residues as long as the peptide retains the requisite ability to associate with NT-3. Substantially similar peptides also include the presence of additional amino acids or the deletion of one or more amino acids which do not affect the requisite ability to associate with NT-3, For example, substantially similar peptides can contain an N- or C—. terminal cysteine, by which, if desired, the peptide may be covalently attached to a carrier protein, e.g., albumin. Such attachment can decrease clearing of the peptide from the blood and also decrease the rate of proteolysis of the peptides. In addition, for purposes of the present disclosure, peptides containing D-amino acids in place of L-amino acids are also included in the term “conservative substitution.” The presence of such D-isomers can help minimize proteolytic activity and clearing of the peptide.

In some embodiments, a pro-neurotrophin-3 protein (pro-NT-3) is administered to the subject. The pro form of neurotrophin-3 is a ~30 kDa precursor form of NT-3 which is converted to the mature NT by enzymatic cleavage and removal of a ~15 kDa N-terminal prodomain. See Tauris et al., Eur. J Neurosci, 33 (4), 622-631 (2011).

### Administration and Formulation

The vector or peptide used with some embodiments of the present disclosure can be incorporated into pharmaceutical compositions suitable for administration to a subject. In some particular embodiments, the pharmaceutical composition comprises the vector of the disclosure and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it can be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the vector or pharmaceutical composition.

The vectors or peptides can be administered acutely (i.e., during the onset or shortly after events leading to muscular atrophy), or can be administered prophylactically (e.g., before scheduled surgery, or before the appearance of signs or symptoms), or administered during the course of muscular atrophy to reduce or ameliorate the progression of symptoms that would otherwise occur. The timing and interval of administration is varied according to the subject's symptoms, and can be administered at an interval of several hours to several days, over a time course of hours, days, weeks or longer, as would be determined by one skilled in the art.

The compositions containing the vectors or peptides are generally administered intravenously. When administered intravenously, the compositions may be combined with other ingredients, such as carriers and/or adjuvants. Peptides may also be covalently attached to a protein carrier, such as albumin, so as to minimize clearing of the peptides. There are no limitations on the nature of the other ingredients, except that such ingredients must be pharmaceutically acceptable, efficacious for their intended administration and cannot degrade the activity of the active ingredients of the compositions.

The pharmaceutical forms suitable for injection include sterile aqueous solutions or dispersions and sterile powders for the extemporaneous preparation of sterile injectable solutions or dispersions. In all cases the ultimate solution form must be sterile and fluid. Typical carriers include a solvent or dispersion medium containing, for example, water buffered aqueous solutions (i.e., biocompatible buffers), ethanol, polyols such as glycerol, propylene glycol, polyethylene glycol, suitable mixtures thereof, surfactants or vegetable oils. Sterilization can be accomplished by any art-recognized technique, including but not limited to, filtration or addition of antibacterial or antifungal agents, for example, paraben, chlorobutanol, phenol, sorbic acid or thimerosal. Further, isotonic agents such as sugars or sodium chloride may be incorporated in the subject compositions.

Production of sterile injectable solutions containing the subject peptides is accomplished by incorporated these compounds in the required amount in the appropriate solvent with various ingredients enumerated above, as required, followed by sterilization, preferably filter sterilization. To obtain a sterile powder, the above solutions are vacuum-dried or freeze-dried as necessary.

When the peptides of the disclosure are administered orally, the pharmaceutical compositions thereof containing an effective dose of the peptide can also contain an inert diluent, as assimilable edible carrier and the like, be in hard or soft shell gelatin capsules, be compressed into tablets, or may be in an elixir, suspension, syrup or the like. The subject peptides are thus compounded for convenient and effective administration in pharmaceutically effective amounts with a suitable pharmaceutically acceptable carrier in a therapeutically effective amount.

The expressions “effective amount” or “therapeutically effective amount,” as used herein, refers to a sufficient amount of agent to stimulate muscle growth or decrease or prevent muscle atrophy. The exact amount required will vary from subject to subject, depending on the species, age, and general condition of the subject, the particular therapeutic agent, its mode and/or route of administration, and the like. It will be understood, however, that the total daily usage of the compounds and compositions of the present disclosure can be decided by an attending physician within the scope of sound medical judgment. The specific therapeutically effective dose level for any particular subject or organism will depend upon a variety of factors including the disorder being treated and the severity of the disorder; the activity of the specific compound employed; the specific composition employed; the age, body weight, general health, sex and diet of the subject; the time of administration, route of administration, and rate of excretion of the specific composition employed; the duration of the treatment; drugs used in combination or coincidental with the specific composition employed; and like factors well known in the medical arts.

The vectors or peptides can be administered in a manner compatible with the dosage formulation and in such amount as well be therapeutically effective. Systemic dosages depend on the age, weight and conditions of the patient and on the administration route. For example, a suitable dose of peptide for the administration to adult humans ranges from about 0.001 to about 20.0 mg per kilogram of body weight. The peptides should preferably be administered in an amount of at least about 50 mg per dose, more preferably in an amount up to about 500 mg to about 1 gram per dose. Since the peptide compositions of this disclosure will eventually be cleared from the bloodstream, re-administration of the compositions is indicated and preferred.

### EXAMPLES

Thus, aspects and embodiments of the disclosure are illustrated by the following examples. However, there are a wide variety of other embodiments within the scope of the present disclosure, which should not be limited to the particular examples provided herein.

### Example 1

### NT-3 Delivery Using scAAV1.tMCK.NTF3

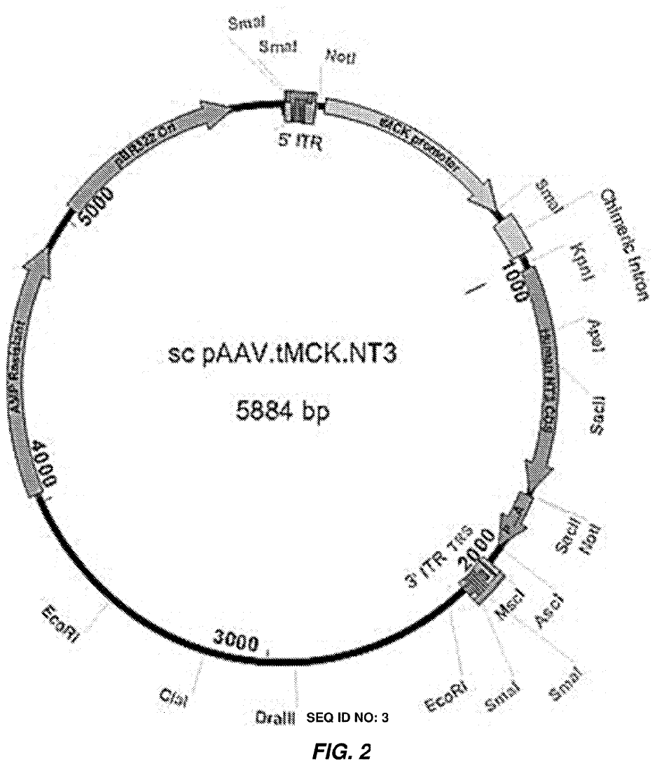

The composition administered is a non-replicating recombinant adeno-associated virus termed scAAV1.tMCK.NTF3, a diagram of the cassette plasmid of which is shown in FIG. 2. The rAAV contains the human NT-3 gene under the control of a tMCK muscle-specific promoter. In vivo biopotency was tested following the intramuscular injection of the rAAV (1×10¹¹ vg) into gastrocnemius muscles of C57B16 mice followed by quantification of circulating NT-3 in the serum by ELISA at 4 to 6 weeks after gene injection.

Firstly, it was demonstrated that scAAV1.CMV.NTF3 delivered to gastrocnemius muscle produced prolonged and therapeutic NT-3 blood levels sufficient to provide functional, electrophysiological and histopathological improvement in TrJ nerves. It was then investigated if it was possible to produce the required rAAV dose and achieve same level of expression by packaging the expression cassette by scAAV1. A dose-response study was performed on C57BL/6 mice comparing serum NT-3 ELISA data following intramuscular injection of scAAV1.tMCK.NTF3 and scAAV1.CMV.NTF3 at 3 doses (3×10⁹ vg, 1×10¹⁰ vg and 3×10¹⁰ vg). Administration of scAAV1.CMV.NTF3 rAAV at 1×10¹¹ vg produced significantly higher NT-3 levels than the single-stranded rAAV at the same dose consistent with greater potency using self-complementary vectors. At a half-log less dose (3×10¹⁰ vg), rAAV comprising either CMV and tMCK produced comparable NT-3 serum levels to those obtained from mice that received scAAV1.CMV.NTF3 at 1×10¹¹ vg dose, which produced a biological response. The NT-3 levels (mean±SEM) were measured from TrJ mice at 24 weeks post injection. There is significant difference in NT-3 levels among all 7 groups, p value<0.0001. A significant difference in NT-3 levels was observed for highest and intermediate doses of rAAVs for both promoters and control. However, analysis failed to find significant difference for lower doses for both rAAV. Kruskal-Wallis test is used to compare serum NT-3 among all groups (PBS, CMV 3E+09/1E+10/3E+10 and tMCK 3E+09/1E+10/3E+10). Mann-Whitney U test is used to compare NT-3 between each group and PBS (control) group, and Bonferroni correction is used to adjust for multiple comparisons. See Sahenk et al., Mol. Ther. 22 (3): 511-521, 2014, which is incorporated by reference herein in its entirety.

Muscle diameter increases at 40 weeks posttreatment: The effects of NT-3 gene therapy was assessed in TrJ mice upon muscle fiber size at 40 weeks postinjection in a subset of animals injected with scAAV1.CMV.NTF3 (1×10¹¹ vg) compared to PBS. Neurogenic changes characterized by atrophic angular fibers and group atrophy were evident in the muscles from untreated mice while evidence for reinnervation as fiber type groupings and an overall fiber size increase were recognizable as treatment effect. Muscle fiber size histograms generated from contralateral anterior and posterior compartment muscles of the left lower limb (tibialis anterior and gastrocnemius) showed an increase in fiber diameter.

Additional studies have shown that NT-3 stimulates Akt/mTOR pathway in SCs cells giving rise to improved myelination and radial growth of axons in the nerve and NT-3 also has a direct stimulatory effect on myotubes through Trk-C receptors indicating its role in fiber diameter increase in muscles of TrJ mice.

Studies with Self-complementary (sc) AAV1 and the Use of a Muscle Specific Truncated Creatine Kinase (tMCK) Promoter

scAAV permits lower dosing that adds up to enhanced safety and dosing levels that will meet production standards. The use of tMCK promoter is a valued objective again offering greater safety by avoiding off target effects. In the following set of experiments the efficacy of scAAV1.NTF3 under control of the CMV promoter was compared to the muscle specific tMCK promoter both given at three doses, within a half-log range (3×10⁹ vg, 1×10¹⁰ vg and 3×10¹⁰ vg). The efficacy AAV1.NTF3 gene transfer in TrJ mice peripheral nerves were assessed by electrophysiological (Table 2) and morphological studies 24 weeks post gene transfer. The evidence of transgene expression was assessed by measuring serum NT-3 levels using ELISA.

TABLE 2
CMAP and Conduction Velocity in the TrJ Sciatic Nerve.

| | | | | | | Conduction |
|---|---|---|---|---|---|---|
| Treatment Group (number) | | Latency (ms) | Duration (ms) | CMAP (mV) | Area (mVms) | Velocity (m/s) |
| PBS | 29 | 2.36 ± 0.08 | 7.00 ± 0.42 | 0.42 ± 0.03* | 0.70 ± 0.09 | 8.24 ± 0.43 |
| AAV1.NTF3.CMV-HD | 23 | 2.43 ± 0.10 | 6.58 ± 0.50 | 0.57 ± 0.04* | 0.84 ± 0.09 | 10.30 ± 0.65 |
| AAV1.NTF3.tMCK-HD | 27 | 2.52 ± 0.07 | 5.64 ± 0.49 | 0.54 ± 0.03** | 0.64 ± 0.04 | 8.90 ± 0.56 |
| AAV1.NTF3.CMV-ID | 21 | 2.08 ± 0.09 | 5.20 ± 0.38 | 0.46 ± 0.04 | 0.61 ± 0.07 | 9.00 ± 0.63 |
| AAV1.NTF3.tMCK-ID | 26 | 2.20 ± 0.08 | 5.85 ± 0.42 | 0.45 ± 0.03 | 0.61 ± 0.06 | 9.01 ± 0.40 |
| AAV1.NTF3.CMV-LD | 22 | 2.23 ± 0.10 | 6.38 ± 0.53 | 0.48 ± 0.04 | 0.68 ± 0.07 | 8.95 ± 0.74 |
| AAV1.NTF3.tMCK-LD | 23 | 2.18 ± 0.06 | 7.29 ± 0.60 | 0.43 ± 0.03 | 0.80 ± 0.08 | 8.40 ± 0.59 |

*p = 0.0017;
**p = 0.0051; Values are Mean ± SEM;
HD = High Dose;
ID = Intermediate Dose;
LD = Low Dose

The examiner during electrodiagnostic studies was blinded to the treatment groups. There is no statistical difference between AAV1.NTF3.CMV (high dose, HD) and AAV1.NTF3.tMCK (high dose, HD) on CMAP and it was preferred to use the muscle specific tMCK promoter. This is further supported by the NT-3 levels in ELISA Assay where a significant difference in NT-3 levels was observed for highest and intermediate doses of rAAV for both promoters and control.

### Example 2

### Construction of NT-3 Expressing AAV Construct

Design of self-complementary rAAV viral vectors with serotype 1 containing NT-3 cDNA under tMCK was described previously in Sahenk et al., Mol Ther, 22 (3): 511-521 (2014), which is incorporated by reference herein in its entirety. Aliquoted viruses were kept in −80° C. until use. Blood samples were collected from treated and non-treated mice by eye bleeding under anesthesia at 6 and 16 weeks post injection and serum was assayed for NT-3 levels using a capture ELISA. The construct is referred to herein as scAAV1.tMCK.NTF3.

A tMCK promoter/enhancer sequence was used to drive muscle-specific gene expression and is composed of the muscle creatine kinase promoter with an added enhancer element (enh358MCK, 584-bp) fused to it. A triple tandem of the MCK enhancer (206-bp) was ligated to the 87-bp basal promoter in the tMCK promoter/enhancer.

The scAAV1.tMCK.NTF3 drug product was produced by 3 plasmid DNA transfection of human HEK293 Master Cell Bank cells with: (i) the pAAV.tMCK.NTF3-vector plasmid (see FIG. 2), (ii) an AAV1 helper plasmid termed R88/C1 containing the AAV rep2 and Cap1 wild-type genes and (iii) the helper adenovirus plasmid.

A schematic representation of the plasmid with molecular features and open reading frames is shown in FIG. 2. The rAAV genome derived from pAAV.tMCK.NTF3 plasmid is a self-complementary DNA genome containing the human NT-3 cDNA expression cassette flanked by AAV2 inverted terminal repeat sequences (ITR). It is this sequence that is encapsulated into AAV1 virions. Plasmid pAAV.tMCK.NTF3 was constructed by inserting the tMCK expression cassette driving a NT-3 gene sequence into the AAV cloning vector psub201. The human NT-3 gene is expressed from the mouse triple tandem MCK promoter which is a modification of the previously described CK6 promoter and contains a triple E box sequence. An SV40 polyadenylation signal is used for efficient transcription termination. The cassette also contains a chimeric intron for increased gene expression and is composed of the 5′ donor site from the first intron of the human β-globin gene and the branchpoint and 3′ splice acceptor site from the intron that is between the leader and the body of an immunoglobulin gene heavy chain variable region. The NT-3 expression cassette has a consensus Kozak immediately in front of the ATG start and 200 bp SV40 polyA signal for efficient mRNA termination. The NT-3 cDNA is included in its entirety (NCBI Reference Sequence: NM_001102654). The only viral sequences included in this vector are the inverted terminal repeats of AAV2, which are required for both viral DNA replication and packaging. The AAV ITRs are sequences that are nearly identical on both ends, but in opposite orientation. The “left” (mutated) ITR has the terminal resolution site deleted to allow hairpin formation of the genome. The identities of all DNA plasmid elements are confirmed by DNA plasmid sequencing on the plasmid source stock.

Shown in Table 3 are the base pair locations of relevant molecular features within the rAAV vector DNA plasmid of SEQ ID NO: 3.

TABLE 3
Molecular Features of plasmid sc pAAV.tMCK.NTF3

| TYPE | START | END | NAME | DESCRIPTION |
|---|---|---|---|---|
| REGION | 7 | 112 | 5′ ITR | AAV2 inverted terminal repeat with |
| | | | | terminal resolution site deleted |
| REGION | 147 | 860 | tMCK promoter/ | Mouse muscle creatine kinase |
| | | | enhancer | promoter/enhancer |
| REGION | 892 | 1024 | chimeric Intron | 5′ donor site from human β-globin and |
| | | | | the branchpoint and 3′ splice acceptor |
| | | | | site from IgG HC variable region. |
| GENE | 1077 | 1850 | Human NT-3 | Human NT-3 gene |
| | | | gene | |
| REGION | 1860 | 2059 | SV40 pA | SV40 polyadenylation signal |
| REGION | 2121 | 2248 | 3′ ITR | Wild-type AAV2 inverted terminal |
| | | | | repeat |
| GENE | 4032 | 4892 | AmpP^{r} | AmpP^{r}P resistance gene |
| REGION | 5040 | 5707 | ori | Plasmid origin of replication |

### Example 3

### Experimental Autoimmune Encephalomyelitis (EAE) Mouse Model to Mimic MS Disease

Experimental autoimmune encephalomyelitis (EAE) is the most commonly used experimental model for the human inflammatory demyelinating disease, multiple sclerosis (MS). Bjelobaba, Ivana, et al. “Animal models of multiple sclerosis: Focus on experimental autoimmune encephalomyelitis.” Journal of Neuroscience Research 96.6 (2018): 1021-1042. EAE is a complex condition in which the interaction between a variety of immunopathological and neuropathological mechanisms leads to an approximation of the key pathological features of MS: inflammation, demyelination, axonal loss, and gliosis. The counter-regulatory mechanisms of resolution of inflammation and remyelination also occur in EAE, which, therefore, can also serve as a model for these processes. EAE has a complex neuropharmacology, and many of the drugs that are in current or imminent use in MS have been developed, tested or validated on the basis of EAE studies.

Chronic relapsing experimental autoimmune encephalomyelitis (EAE) mouse model was used to mimic MS disease progression and clinical status in human patients in this disclosure. EAE model is the most common and widely accepted mouse model to mimic MS, as they share histopathologic and immunologic similarities. The efficacy of AAV NT-3 gene therapy approach to treat EAE mouse as an indicator for using this approach to treat MS was investigated.

The Examples provided in the present disclosure investigate the possibility of using NT-3 as a treatment approach for treating multiple sclerosis and other autoimmune diseases. Preliminary data shows that AAV1.tMCK.NT-3 gene therapy ameliorated the clinical severity of EAE, without wishing to be bound by theory, possibly by modulating the immune system. The possibility of using AAV1.tMCK.NT-3 gene therapy, via intramuscular delivery into the EAE model to attenuate the disease process may prove to be a useful treatment approach. Overall, NT-3 showed potential of modulating immune system, thus has the potential to be used as treatment for chronic progressive MS and potentially for other autoimmune diseases.

### Example 4

Treatment with NT-3 Gene Therapy Improved Clinical Score and Behavioral Outcomes in EAE Mice

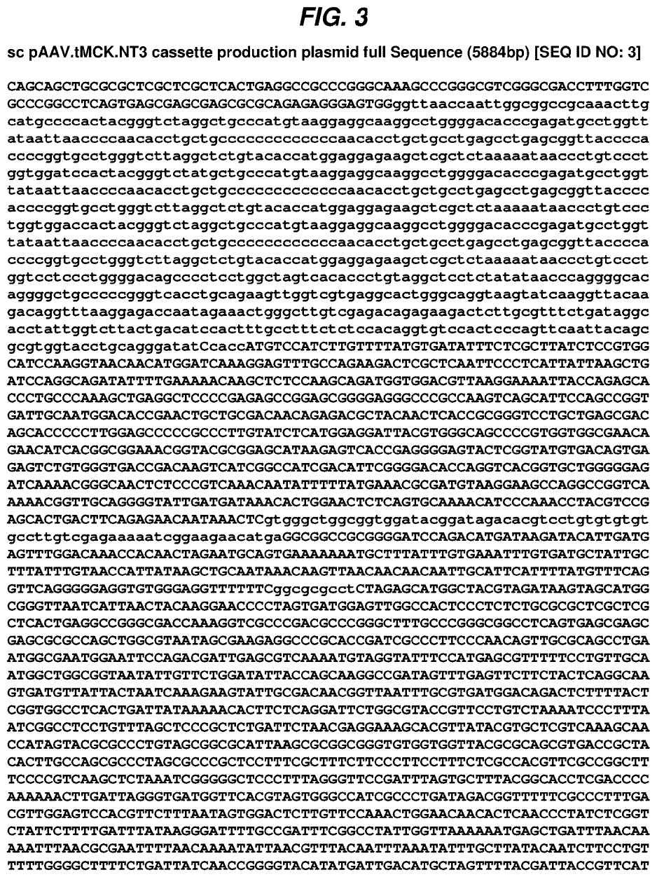

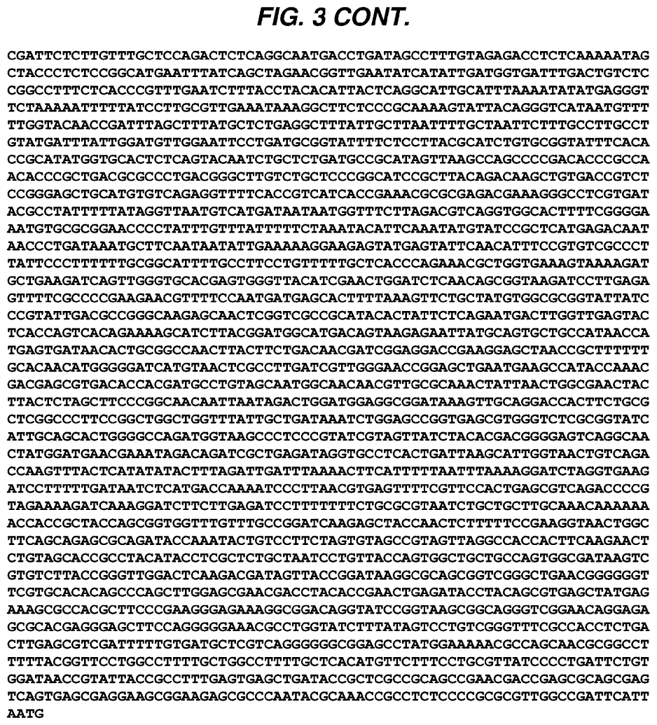

The induction of EAE was carried out using the protocol published by Bittner, Stefan, et al. “Myelin oligodendrocyte glycoprotein (MOG35-55) induced experimental autoimmune encephalomyelitis (EAE) in C57BL/6 mice.” Journal of Visualized Experiments: JoVE 86 (2014). Briefly, both male (n=4) and female (n=5) C57BL/6 mice at age of 8-12 weeks were immunized with synthetic myelin oligodendrocyte glycoprotein peptide 35-55 (MOG₃₅₋₅₅, MEVGWYRSPFSRVVHLYRNGK) in complete Freund's adjuvant (CFA) with 4 mg/mL Mycobacterium tuberculosis H37RA. A total of 200 μg MOG₃₅₋₅₅ peptides and 200 μg H37RA was emulsified in CFA and injected subcutaneously (sc) into the flanks of the mice. Mice were also intraperitoneally (IP) injected with 400 ng of pertussis toxin in 200 μl of PBS. A second dose of pertussis toxin was given after 48 hrs. AAV1.tMCK.NTF3 was delivered via intramuscular (IM) injection to the right gastrocnemius muscle (1.0×¹¹ vg in Ringer's lactate, 50 μl volume) at 21 days post EAE induction (one week after peak disease activity) and mice were closely monitored and sacrificed at around 70 days post EAE induction. EAE induction resulted in the expected peak of disease activity at 2 weeks; therefore, to assess treatment efficacy during the chronic phase, NT-3 gene therapy was delivered 1 week after the peak disease activity, at the plateau phase of the disease progression. This approach ensures systemic NT-3 effect through secretion from muscle into circulation. Endpoint blood samples from terminally anesthetized EAE-induced and untreated mice were collected by cardiac puncture, and serum was assayed for NT-3 levels using a capture ELISA as previously reported in Yalvac et al. (Mol. Ther. 22:1353-1363, 2014). The serum NT-3 in the AAV1.NT-3 treated mice easily were detected, whereas the levels in untreated cohort was below the assay's detection threshold (FIG. 3).

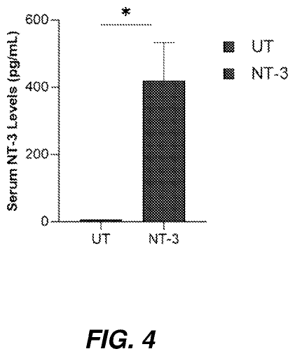

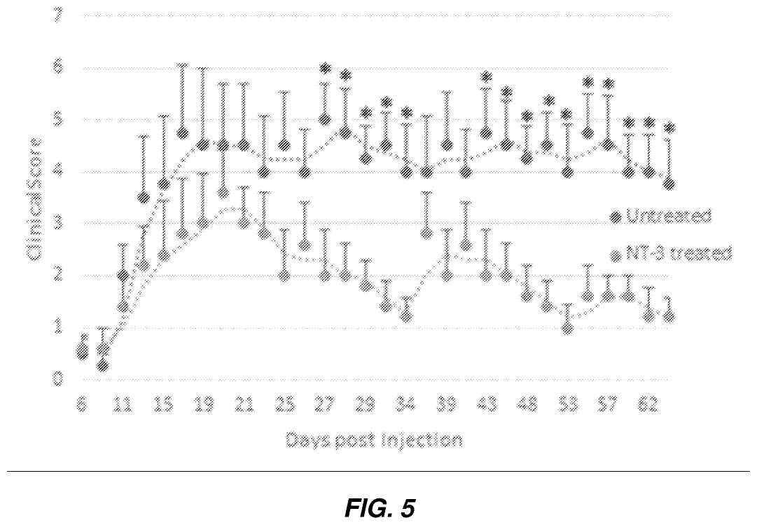

The mice were monitored every other day after the EAE induction. The clinical score of each mouse were defined as set out in Table 3, with 0 indicating no clinical signs and 10 indicating death (Terry et al., Methods Mol Biol; 1304:145-160, 2016). The higher scores indicate more severe symptoms and thereby a worse clinical status of the mouse. Overall, females were more severely affected than males (data not shown). Males and females combined, preliminary data indicated that NT-3 treatment significantly reduced the clinical score of the mice, thus ameliorated the severity of EAE (FIG. 4). The clinical score showed substantial improvement of treated mice compared to untreated mice.

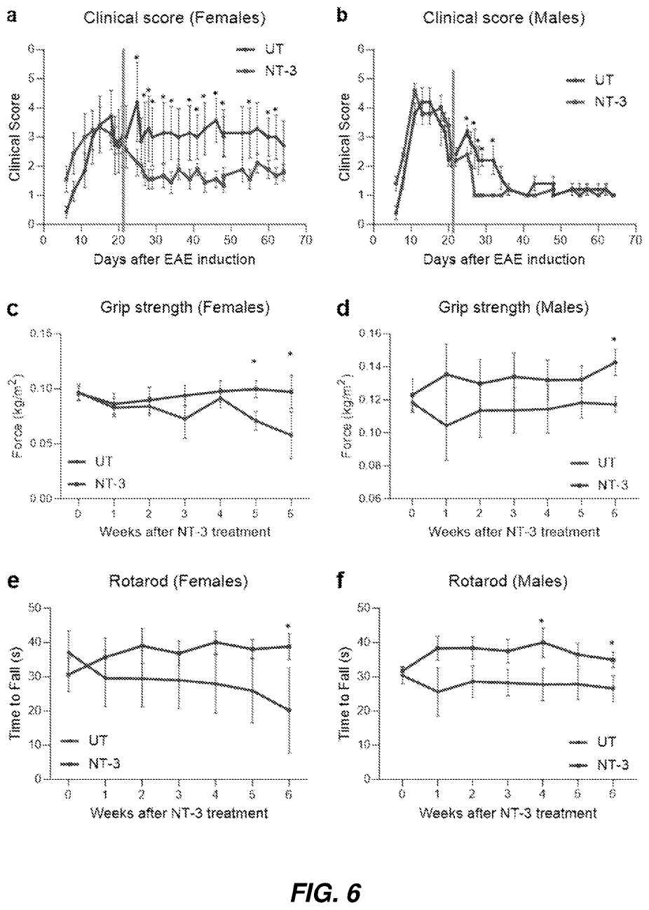

Additional studies also showed a significant and steady reduction from the clinical score of an average of 3 at day 15 to an average of 1.5 by day 30 and afterwards was observed, and the difference with the untreated cohort was statistically significant between 25-70 days post EAE induction. In the male cohort, a significant clinical score reduction was observed in the treated group, within the 25 to 35 days post EAE induction range, although no further improvement was observed after 40 days (FIG. 6b).

TABLE 3
Clinical score guideline for EAE model.

| Score | Clinical signs |
|---|---|
| 0 | No clinical signs |
| 1 | Partially loss of tail tonus |
| 2 | Paralyzed tail |
| 3 | Moderate hind limb paraparesis |
| 4 | Severe hind limb paraparesis |
| 5 | Partial hind limb paralysis |
| 6 | Hind limb paralysis |
| 7 | Hind limb paralysis; one forelimb paralysis |
| 8 | Hind limbs paralyzed, both forelimbs paralyzed |
| 9 | Moribund |
| 10 | Death |

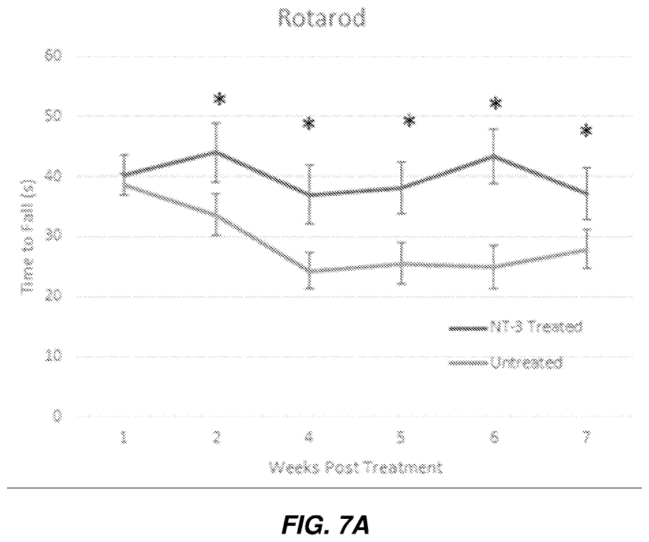

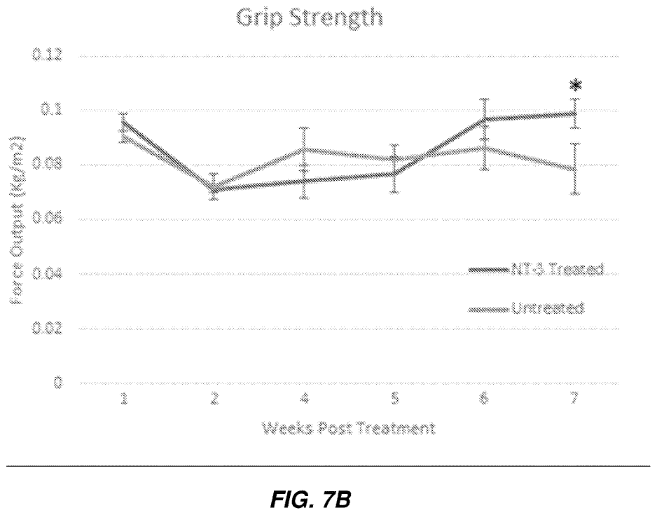

In addition to clinical score, behavior tests were carried out in the EAE mice (FIG. 7). The rotarod data (FIG. 7A) indicated that the NT-3 treated cohort displayed significantly better motor coordination compared to the untreated group. As shown in FIG. 7B, grip strength of the treated mice showed little change until about 5.5 weeks post NT-3 treatment, and treated cohort started showing the trend of increasing in grip strength afterwards. These data correlated with reduced expression of the pro-inflammatory cytokines, interleukin-1β (IL-1β), tumor necrosis factor (TNF-α), and interleukin-6 (IL-6) in both brain and spinal cord, in the NT-3 treated cohort, as shown in Example 5.

Additional studies are in agreement with improvements in the clinical scores, the treated EAE cohorts performed significantly better in grip strength (FIG. 6c, d) and rotarod tests (FIG. 6e, f), compared to untreated counterparts. To investigate the functional outcome of treatment in the EAE mice, grip strength for hind limb and rotarod functions were assessed weekly, starting from the day of AAV1.NTF3 injection (3 weeks post EAE induction). The grip strength force of AAV1.NTF3 treated females (FIG. 6c) stayed relatively stable over a six-week period (mean of 0.095 kg/m2 at week 0 to 0.097 kg/m2 at week 6). In contrast, a decline of this function was observed in the untreated female cohort by approximately 40% (from mean of 0.096 kg/m² to 0.058 kg/m² at week 6) within the same period, resulting significantly higher force generation in the treated compared to the untreated counterparts at week 5 (NT-3:0.10±0.008 kg/m², n=7 vs. UT: 0.071±0.009 kg/m²; p=0.027) and 6 (NT-3:0.097±0.015 kg/m² vs. UT: 0.058±0.021 kg/m²; p=0.032). Males showed higher grip strength performance than female counterparts at the baseline (FIG. 6d). For the untreated male cohort, the grip strength remained relatively stable (mean of 0.118 kg/m² at week 0 and mean of 0.117 kg/m² at week 6); whereas AAV.NTF3 treated cohort showed a slight increase of performance, of about 14% (mean of 0.123 kg/m² to mean of 0.142 kg/m²). Similar to females, AAV1.NTF3 treatment in males resulted in an overall higher grip strength, starting 1 week post gene delivery compared to their untreated counterparts, which became statistically significant at week 6 (NT-3:0.143±0.008 kg/m² vs UT: 0.117±0.005 kg/m² n=5 per cohort; p=0.023).

In rotarod testing, the treated female cohort (FIG. 6e) showed an overall better performance between week 1 through 6 post treatment while a gradual decline was present in the untreated controls, giving rise to a 48% higher performance with treatment at week 6 (38.8±3.8 s vs. 20.2±12.5 s, p=0.02). The treatment response in rotarod performance of males was more subdued (FIG. 6f), showing significance at week 4 and 6 (40.0±4.3 s vs.27.7±4.7 s in week 4, p=0.045 and 34.9±2.2 s vs. 26.6±3.7 s in week 6, p=0.047). At endpoint, the untreated cohort showed 13% decrease in rotarod time from the baseline (30.3 s at week 0 to 26.6 s at week 6), whereas in the AAV1.NTF3 treated cohort the rotarod time increased by 10% compared to baseline (from 31.7s at week 0 to 34.9s at week 6), corresponding to a 23.8% increase at endpoint due to treatment, compared to the untreated counterparts. Collectively these data show that AAV1.NTF3 gene therapy in the EAE mouse results in significant improvements in clinical scores, with more strength in hindlimb grip testing, and better sensorimotor function on rotarod performance. It should be noted that NT-3 effect in both sexes is equal, normalization toward WT, and that the greater percent change in functional tests or clinical scores in females are related to the disease course, being more severe in females than males.

### Example 5

### AAV1.NTF3 Treated EAE Mice Show Reduced Inflammation, Improved Remyelination and Axon Protection in Spinal Cord

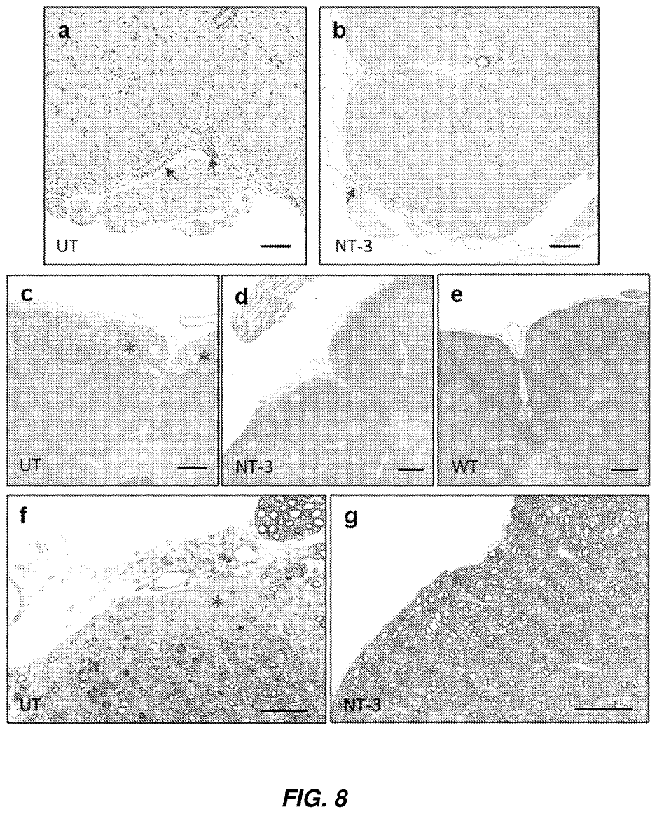

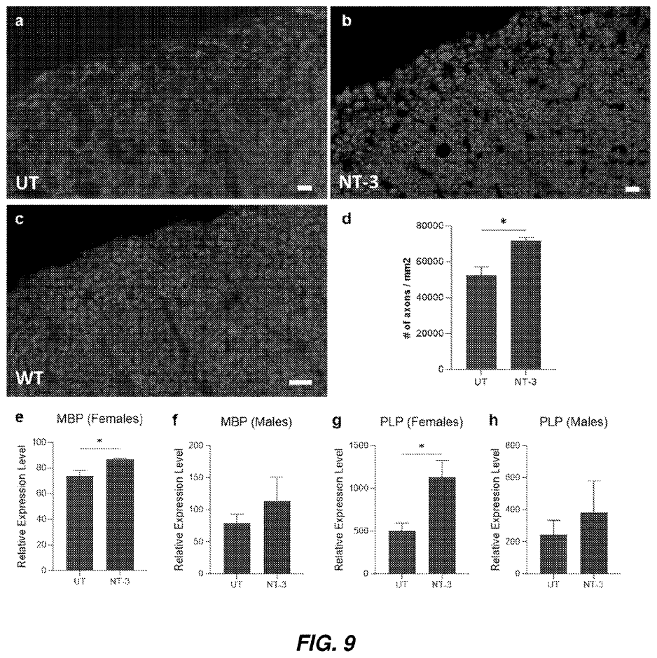

Hematoxylin & Eosin (H&E) stained cross sections from sacral, lumbar, mid, and upper thoracic spinal cord segments of untreated and treated mice (n=4 per cohort) at 10 weeks post EAE induction were examined to assess inflammation. Multifocal perivascular subpial inflammation was present in all untreated mice at all four different levels of spinal cord segments examined (FIG. 8a). In the AAV1.NTF3 treated group (FIG. 8b), only a few small areas of perivascular inflammation were observed in one out of four segments from two mice. Moreover, Luxor fast blue (LFB) stained paraffin sections (FIG. 8c-e) and toluidine blue stained semi thick plastic sections from treated mice revealed preservation of the white matter long tracts as illustrated in the descending anterolateral corticospinal tract at 7 weeks post treatment (FIG. 8f, g). Specifically, both preserved myelinated fibers and abundant thinly remyelinated axons were observed with treatment in the high-resolution plastic embedded sections. Moreover, quantitative immunofluorescence studies using an anti-neurofilament antibody as an axonal marker revealed that AAV1.NTF3 gene therapy significantly attenuated axon loss within the long tracts of the spinal cord (FIG. 9a-d). We also showed that improved remyelination and axon protection was clearly associated with NT-3-induced increases in the expression levels of myelin basic protein (MBP) and proteolipid protein (PLP) (FIG. 9e-h). This emphasizes the NT-3 effect as increase of remyelination and its protective effect on myelinated fiber integrity. Collectively, these observations indicate NT-3 gene therapy significantly decreased subpial inflammation, improved remyelination and reduced axon loss in the white matter of the spinal cord. No pathologic changes were detected in the roots, sciatic nerves, or muscles. Example 6 Characterization of the Immunododulatory Effect of NT-3 Gene Therapy in EAE Mice

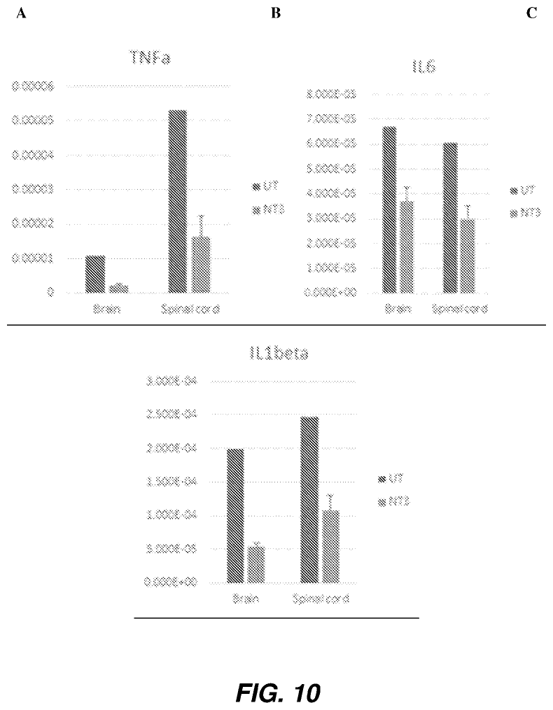

Since EAE is a chronic autoinflammatory disease affecting brain and spinal cord, the inflammatory status in mice in these tissues was investigated. The levels of inflammatory markers TNFα, IL1B, and IL6 expression in the brains and spinal cord of treated and untreated mice were measured. Experimental data collected indicated that all three inflammation markers were significantly reduced in the NT-3 treated cohort (FIG. 10).

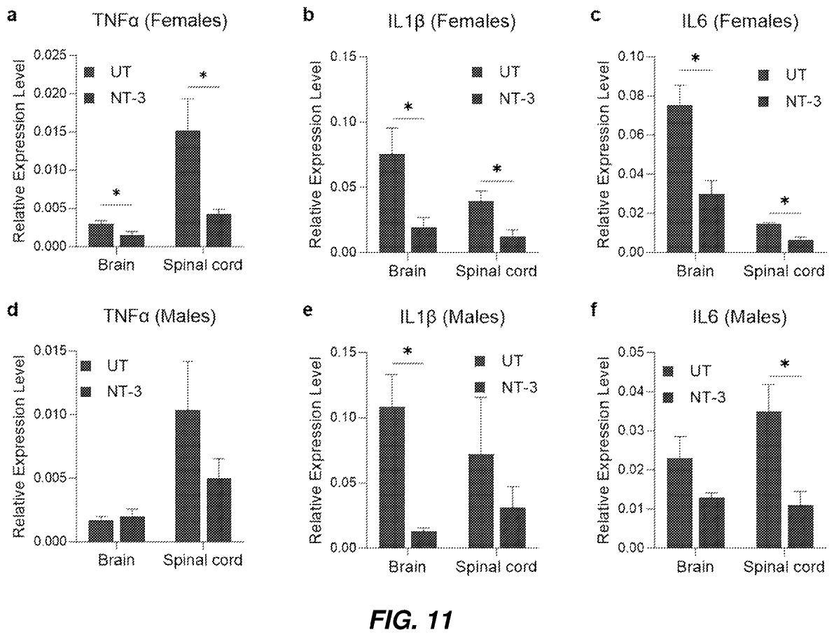

In additional studies to characterize the immunomodulatory effect of treatment, RNA was isolated from fresh frozen brain and spinal cord tissue samples, and real time-PCR was performed to determine the expression level of inflammatory markers, including TNFα, IL1β and IL6. In the female cohort, the NT-3-treated group exhibited significantly reduced expression levels of all three markers in both brain and spinal cord compared to samples from untreated EAE mice (FIG. 11a-c). The male cohort, manifesting a milder disease, displayed less pronounced treatment efficacy. The AAV1.NTF3 gene delivery in the male EAE mice resulted in a significantly reduced IL1B expression in brain, and IL6 in spinal cord, while TNFα and IL1B expression levels in the spinal cord, and IL6 levels in brain decreased without reaching significance level (FIG. 11d-f).

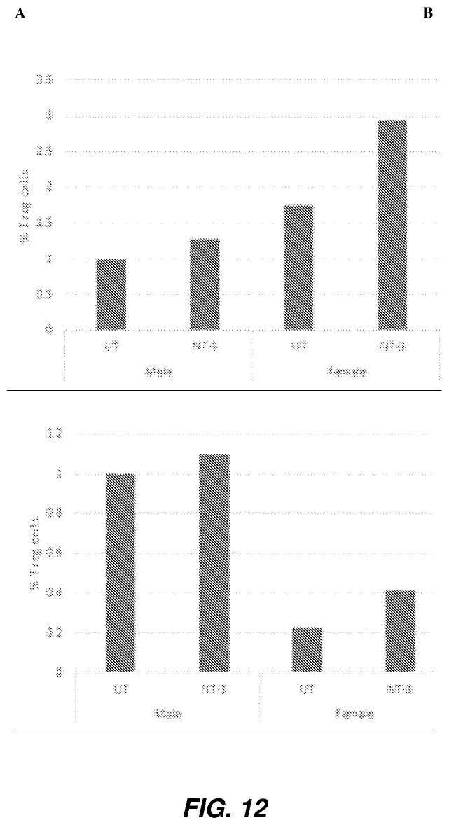

Regulatory T cells (Treg cells) are thought to play a critical role in the maintenance of peripheral immune tolerance. It is believed that Tregs operate by suppressing the effector CD4+T cell subsets that mediate autoimmune responses. Dysregulation of suppressive and migratory markers on Tregs have been linked to the pathogenesis of MS37. To investigate whether AAV1.NTF3 treatment increased the percentage of CD4+CD25+Foxp3+Tregs cells in the lymph nodes and spleen from the treated and untreated EAE mice was measured. Treg cells commonly serve as an immunosuppressor and are critical in preventing autoimmune disease. As shown in FIG. 12, NT-3 gene therapy increased the percentage of CD3+CD4+CD25+ and Foxp3+ Treg cells in total cells extracted from lymph node and spleen in both male and female EAE mice indicating an immunomodulatory effect of NT-3. Since MS patients are known to be persistently low in Treg cells, the observed immunomodulatory effect of NT-3 in the EAE model is significant, and suggests the potential for efficacy of NT-3 gene therapy in chronic progressive MS cases in humans.

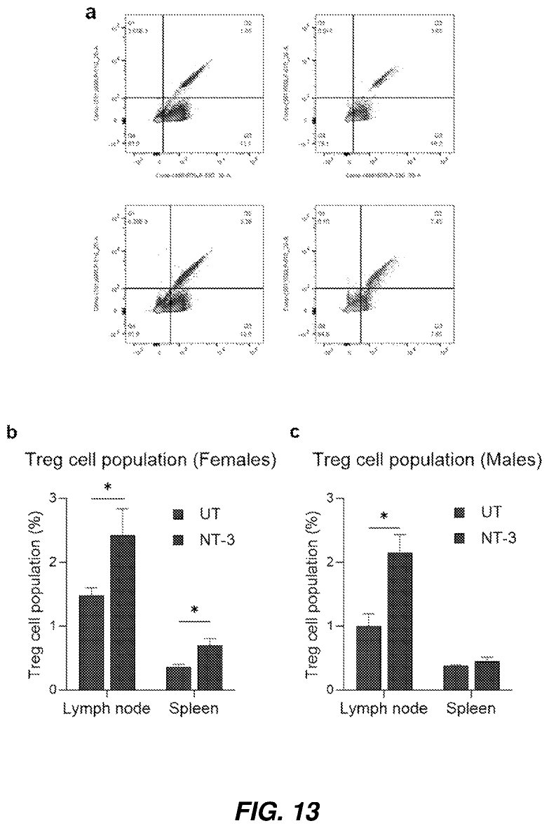

In an additional study, the percentage of CD4+CD25+Foxp3+Tregs cells in the EAE mice, was analyzed in the cell population from lymph node and spleen by flow cytometry (FIG. 13a) after gene therapy treatment. AAV1.NTF3 treatment in both female and male cohorts significantly increased the percentage of Treg cell population in lymph nodes compared to samples from untreated counterparts (FIG. 13b, c). We also found a notable increase of Treg cell population in splenocytes (approximately 52%) from treated females (FIG. 13b) while no significant increase was present in males (FIG. 13c). These results are compatible with an immune-regulatory role of NT-3, in favor of attenuation of the severity of EAE.

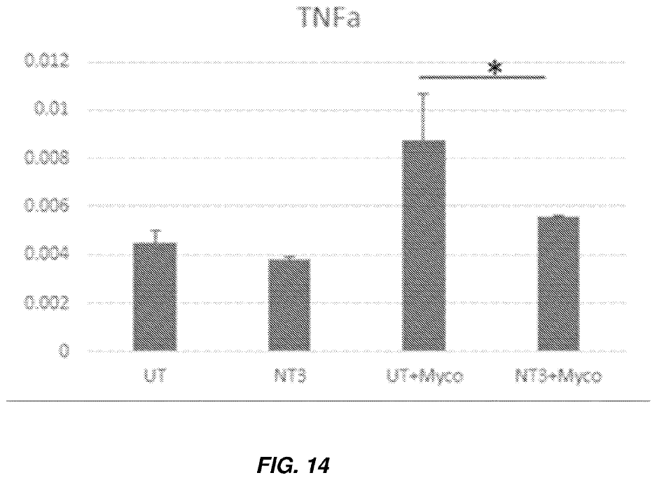

Circulating bone marrow-derived dendritic cells (DCs) are crucial antigen-(Ag-) presenting cells (APCs) that accumulate in the CNS during EAE (Clarkson et al., Journal of neuroimmunology; 277:39-4, 2014). These migratory cells can present major histocompatibility complex (MHC)-restricted Ag to CD4+ T cells and CD8+ T cells (cross presentation) and thus play central roles in priming adaptive immune responses. To investigate whether NT-3 treatment caused dendritic cells (DCs) to gain a tolerogenic feature, DCs from the bone marrow of treated and untreated EAE mice was extracted. The cultured DCs were challenged with mycobacterium for 24 hours, and subsequently the level of TNFα was determined as a marker for inflammation. After 24 hours of incubation with mycobacterium, DCs from NT-3 treated cohort demonstrated lower levels of TNFα (i.e., lower induction of TNFα) compared to the untreated cohort (FIG. 14).

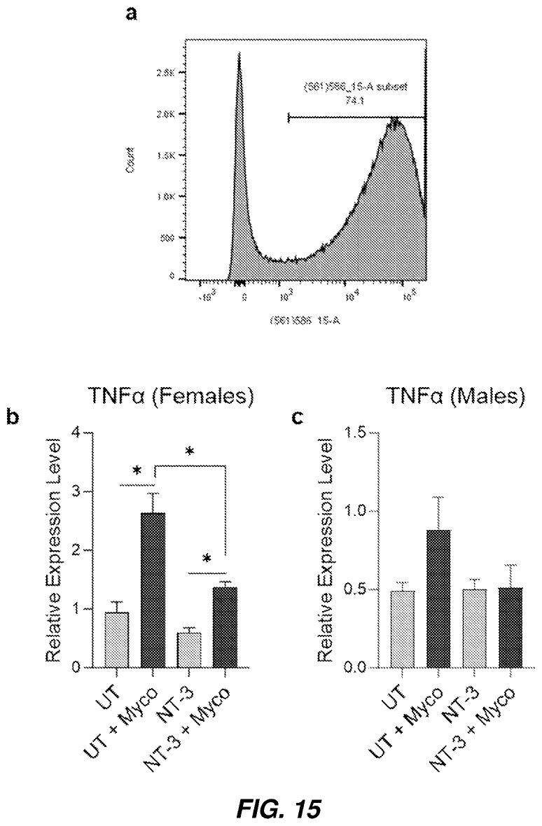

In an additional study, bone marrow DCs were isolated from the femur of AAV1.NTF3 treated untreated cohorts and the population of DCs were determined by positive signal from DC marker CD11c. We first determined that approximately 74.1% of the total cells analyzed were positive for CD11c marker (FIG. 15a). To investigate whether AAV1.NTF3 treatment causes DCs to gain a tolerogenic feature, bone marrow-derived DCs from treated and untreated cohorts was exposed to Mycobacteria. DCs from untreated females at 24 hours of incubation with Mycobacteria showed a significant increase of the proinflammatory marker TNFα expression by qPCR (FIG. 15b). However, this TNFα increase was significantly lower in DCs exposed to Mycobacteria from the AAV1.NTF3 treated group compared with the untreated counterpart indicating that NT-3 has potential to induce tolerogenic characteristics in DCs. DCs from the untreated males also showed an increase of TNFα levels in response to Mycobacteria challenge without reaching statistical significance (FIG. 15c), although no changes were observed in the TNFα levels from the treated group at 6 weeks post gene injection. Overall, these results support an immunoregulatory role of NT-3 in favor of tolerogenicity providing neuroprotection and safety measures against the EAE disease process.

### CONCLUSION

The data provided demonstrates that NT-3 has an immunoregulatory role in modulating the autoimmune system in the EAE mouse model of MS. NT-3 treatment successfully ameliorated the clinical severity of EAE in the mouse model by regulating its immune system. AAV1.NTF3 gene therapy reduced the severity of EAE model of MS, possibly through modulation of the immune system.

The data shows that circulating NT-3, following the transduction of muscle via AAV1.tMCK.NTF3 vector injection triggers tolerogenic immune responses by reducing the inflammation and increasing Treg cells in both spleen and lymph node. This proof-of-principle study demonstrates the potential of clinical translational path for AAV-delivered NT-3 for treatment of chronic progressive MS.

The complete disclosure of all patents, patent applications, and publications, and electronically available material cited herein are incorporated by reference. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

While the present disclosure has been described in terms of specific embodiments, it is understood that variations and modifications will occur to those skilled in the art. Accordingly, only such limitations as appear in the claims should be placed on the disclosure.

### REFERENCES

1. Rodriguez Murua S, Farez M F, Quintana F J. The Immune Response in Multiple Sclerosis. Annu Rev Pathol 2022; 17:121-139.
2. Dendrou C A, Fugger L, Friese M A. Immunopathology of multiple sclerosis. Nat Rev Immunol 2015; 15:545-558.
3. Trapp B D, Peterson J, Ransohoff R M, Rudick R, Mork S, Bo L. Axonal transection in the lesions of multiple sclerosis. N Engl J Med 1998; 338:278-285.
4. Wujek J R, Bjartmar C, Richer E, Ransohoff R M, Yu M, Tuohy V K, Trapp B D. Axon loss in the spinal cord determines permanent neurological disability in an animal model of multiple sclerosis. J Neuropathol Exp Neurol 2002; 61:23-32.
5. Dutta R, Trapp B D. Pathogenesis of axonal and neuronal damage in multiple sclerosis. Neurology 2007; 68: S22-31; discussion S43-54.
6. Bebo B, Cintina I, LaRocca N, Ritter L, Talente B, Hartung D, Ngorsuraches S, Wallin M, Yang G. The Economic Burden of Multiple Sclerosis in the United States: Estimate of Direct and Indirect Costs. Neurology 2022; 98: e1810-e1817.
7. Segal B M. Stage-specific immune dysregulation in multiple sclerosis. Journal of interferon & cytokine research: the official journal of the International Society for Interferon and Cytokine Research 2014; 34:633-640.
8. Hu D, Notarbartolo S, Croonenborghs T, Patel B, Cialic R, Yang T H, Aschenbrenner D, Andersson K M, Gattorno M, Pham M, Kivisakk P, Pierre I V, Lee Y, et al. Transcriptional signature of human pro-inflammatory T (H) 17 cells identifies reduced IL10 gene expression in multiple sclerosis. Nature communications 2017; 8:1600.
9. Mahad D H, Trapp B D, Lassmann H. Pathological mechanisms in progressive multiple sclerosis. Lancet Neurol 2015; 14:183-193.
10. Hauser S L, Cree B A C. Treatment of Multiple Sclerosis: A Review. The American journal of medicine 2020; 133:1380-1390 e1382.
11. Segal B M. Modulation of the Innate Immune System: A Future Approach to the Treatment of Neurological Disease. Clin Immunol 2018; 189:1-3.
12. Barbacid M. The Trk family of neurotrophin receptors. J Neurobiol 1994; 25:1386-1403.
13. Huang E J, Wilkinson G A, Farinas I, Backus C, Zang K, Wong S L, Reichardt L F. Expression of Trk receptors in the developing mouse trigeminal ganglion: in vivo evidence for NT-3 activation of TrkA and TrkB in addition to TrkC. Development (Cambridge, England) 1999; 126:2191-2203.
14. Sahenk Z. Neurotrophins and peripheral neuropathies. Brain Pathol 2006; 16:311-319.
15. Richner M, Ulrichsen M, Elmegaard S L, Dieu R, Pallesen L T, Vaegter C B. Peripheral nerve injury modulates neurotrophin signaling in the peripheral and central nervous system. Molecular neurobiology 2014; 50:945-970.
16. Meier C, Parmantier E, Brennan A, Mirsky R, Jessen K R. Developing Schwann cells acquire the ability to survive without axons by establishing an autocrine circuit involving insulin-like growth factor, neurotrophin-3, and platelet-derived growth factor-BB. J Neurosci 1999; 19:3847-3859.
17. Sekimoto M, Tsuji T, Matsuzaki J, Chamoto K, Koda T, Nemoto K, Degawa M, Nishimura S, Nishimura T. Functional expression of the TrkC gene, encoding a high affinity receptor for NT-3, in antigen-specific T helper type 2 (Th2) cells. Immunology letters 2003; 88:221-226.
18. Yalvac M E, Arnold W D, Braganza C, Chen L, Mendell J R, Sahenk Z. AAV1.NT-3 gene therapy attenuates spontaneous autoimmune peripheral polyneuropathy. Gene therapy 2015.
19. Sahenk Z, Galloway G, Clark K R, Malik V, Rodino-Klapac L R, Kaspar B K, Chen L, Braganza C, Montgomery C, Mendell J R. AAV1.NT-3 gene therapy for charcot-marie-tooth neuropathy. Mol Ther 2014; 22:511-521.
20. Yalvac M E, Amornvit J, Chen L, Shontz K M, Lewis S, Sahenk Z. AAV1.NT-3 gene therapy increases muscle fiber diameter through activation of mTOR pathway and metabolic remodeling in a CMT mouse model. Gene therapy 2018.
21. Ozes B, Myers M, Moss K, Mckinney J, Ridgley A, Chen L, Bai S, Abrams C K, Freidin M M, Mendell J R, Sahenk Z. AAV1.NT-3 gene therapy for X-linked Charcot-Marie-Tooth neuropathy type 1. Gene therapy 2021.
22. Ozes B, Moss K, Myers M, Ridgley A, Chen L, Murrey D, Sahenk Z. AAV1.NT-3 gene therapy in a CMT2D model: phenotypic improvements in Gars (P278KY/+) mice. Brain Commun 2021; 3: fcab252.
23. Ozes B, Tong L, Myers M, Moss K, Ridgley A, Sahenk Z. AAV1.NT-3 gene therapy prevents age-related sarcopenia. Aging (Albany NY) 2023; 15:1306-1329.
24. Sahenk Z, Nagaraja H N, McCracken B S, King W M, Freimer M L, Cedarbaum J M, Mendell J R. NT-3 promotes nerve regeneration and sensory improvement in CMT1A mouse models and in patients. Neurology 2005; 65:681-689.
25. Beutner C, Lepperhof V, Dann A, Linnartz-Gerlach B, Litwak S, Napoli I, Prinz M, Neumann H. Engineered stem cell-derived microglia as therapeutic vehicle for experimental autoimmune encephalomyelitis. Gene Ther 2013; 20:797-806.
26. Barres B A, Raff M C, Gaese F, Bartke I, Dechant G, Barde Y A. A crucial role for neurotrophin-3 in oligodendrocyte development. Nature 1994; 367:371-375.
27. Coelho R P, Yuelling L M, Fuss B, Sato-Bigbee C. Neurotrophin-3 targets the translational initiation machinery in oligodendrocytes. Glia 2009; 57:1754-1764.
28. Barres B A, Schmid R, Sendnter M, Raff M C. Multiple extracellular signals are required for long-term oligodendrocyte survival. Development (Cambridge, England) 1993; 118:283-295.
29. Miller S D, Karpus W J. Experimental autoimmune encephalomyelitis in the mouse. Curr Protoc Immunol 2007; Chapter 15:15 11 11-15 11 18.
30. Khan N, Gordon R, Woodruff T M, Smith M T. Antiallodynic effects of alpha lipoic acid in an optimized RR-EAE mouse model of MS-neuropathic pain are accompanied by attenuation of upregulated BDNF-TrkB-ERK signaling in the dorsal horn of the spinal cord. Pharmacology research & perspectives 2015; 3: e00137.
31. Attia Z, Rowe J C, Kim E, Varikuti S, Steiner H E, Zaghawa A, Hassan H, Cormet-Boyaka E, Satoskar A R, Boyaka P N. Inhibitors of elastase stimulate murine B lymphocyte differentiation into IgG- and IgA-producing cells. Eur J Immunol 2018; 48:1295-1301.
32. Kim E, Attia Z, Woodfint R M, Zeng C, Kim S H, Steiner H E, Shukla R K, Liyanage N P M, Ghimire S, Li J, Renukaradhya G J, Satoskar A R, Amer A O, et al. Inhibition of elastase enhances the adjuvanticity of alum and promotes anti-SARS-COV-2 systemic and mucosal immunity. Proc Natl Acad Sci USA 2021; 118.
33. Yalvac M E, Arnold W D, Hussain S R, Braganza C, Shontz K M, Clark K R, Walker C M, Ubogu E E, Mendell J R, Sahenk Z. VIP-expressing dendritic cells protect against spontaneous autoimmune peripheral polyneuropathy. Mol Ther 2014; 22:1353-1363.
34. Terry R L, Ifergan I, Miller S D. Experimental Autoimmune Encephalomyelitis in Mice. Methods Mol Biol 2016; 1304:145-160.
35. Giralt M, Molinero A, Hidalgo J. Active Induction of Experimental Autoimmune Encephalomyelitis (EAE) with MOG (35-55) in the Mouse. Methods Mol Biol 2018; 1791:227-232.
36. Bordon Y. Autoimmunity: A breakthrough to explain sex bias? Nat Rev Immunol 2014; 14:355.
37. Danikowski K M, Jayaraman S, Prabhakar B S. Regulatory T cells in multiple sclerosis and myasthenia gravis. J Neuroinflammation 2017; 14:117.
38. Clarkson B D, Walker A, Harris M, Rayasam A, Sandor M, Fabry Z. Mapping the accumulation of co-infiltrating CNS dendritic cells and encephalitogenic T cells during EAE. Journal of neuroimmunology 2014; 277:39-49.
39. Greter M, Heppner F L, Lemos M P, Odermatt B M, Goebels N, Laufer T, Noelle R J, Becher B. Dendritic cells permit immune invasion of the CNS in an animal model of multiple sclerosis. Nature medicine 2005; 11:328-334.
40. Haase S, Linker R A. Inflammation in multiple sclerosis. Therapeutic advances in neurological disorders 2021; 14:17562864211007687.
41. Lassmann H. Pathogenic Mechanisms Associated With Different Clinical Courses of Multiple Sclerosis. Front Immunol 2018; 9:3116.
42. Nikic I, Merkler D, Sorbara C, Brinkoetter M, Kreutzfeldt M, Bareyre F M, Bruck W, Bishop D, Misgeld T, Kerschensteiner M. A reversible form of axon damage in experimental autoimmune encephalomyelitis and multiple sclerosis. Nature medicine 2011; 17:495-499.
43. Trapp B D, Vignos M, Dudman J, Chang A, Fisher E, Staugaitis S M, Battapady H, Mork S, Ontaneda D, Jones S E, Fox R J, Chen J, Nakamura K, et al. Cortical neuronal densities and cerebral white matter demyelination in multiple sclerosis: a retrospective study. Lancet Neurol 2018; 17:870-884.
44. Mey G M, Mahajan K R, DeSilva T M. Neurodegeneration in multiple sclerosis. WIREs Mech Dis 2023; 15: e1583.
45. Losseff N A, Wang L, Lai H M, Yoo D S, Gawne-Cain M L, McDonald W I, Miller D H, Thompson A J. Progressive cerebral atrophy in multiple sclerosis. A serial MRI study. Brain 1996; 119 (Pt 6): 2009-2019.
46. Lublin F D, Reingold S C, Cohen J A, Cutter G R, Sorensen P S, Thompson A J, Wolinsky J S, Balcer L J, Banwell B, Barkhof F, Bebo B, Jr., Calabresi P A, Clanet M, et al. Defining the clinical course of multiple sclerosis: the 2013 revisions. Neurology 2014; 83:278-286.
47. Antel J, Antel S, Caramanos Z, Arnold D L, Kuhlmann T. Primary progressive multiple sclerosis: part of the MS disease spectrum or separate disease entity? Acta Neuropathol 2012; 123:627-638.
48. Rovaris M, Confavreux C, Furlan R, Kappos L, Comi G, Filippi M. Secondary progressive multiple sclerosis: current knowledge and future challenges. Lancet Neurol 2006; 5:343-354.
49. Haque A, Trager N N M, Butler J T, Das A, Zaman V, Banik N L. A novel combination approach to effectively reduce inflammation and neurodegeneration in multiple sclerosis models. Neurochem Int 2024; 175:105697.
50. Christodoulou M V, Petkou E, Atzemoglou N, Gkorla E, Karamitrou A, Simos Y V, Bellos S, Bekiari C, Kouklis P, Konitsiotis S, Vezyraki P, Peschos D, Tsamis K I. Cell replacement therapy with stem cells in multiple sclerosis, a systematic review. Hum Cell 2024; 37:9-53.
51. Karavanov A, Sainio K, Palgi J, Saarma M, Saxen L, Sariola H. Neurotrophin 3 rescues neuronal precursors from apoptosis and promotes neuronal differentiation in the embryonic metanephric kidney. Proc Natl Acad Sci USA 1995; 92:11279-11283.
52. Nakajima K, Kikuchi Y, Ikoma E, Honda S, Ishikawa M, Liu Y, Kohsaka S. Neurotrophins regulate the function of cultured microglia. Glia 1998; 24:272-289.
53. Tzeng S F, Huang H Y. Downregulation of inducible nitric oxide synthetase by neurotrophin-3 in microglia. J Cell Biochem 2003; 90:227-233.
54. Tzeng S F, Huang H Y, Lee T I, Jwo J K. Inhibition of lipopolysaccharide-induced microglial activation by preexposure to neurotrophin-3. J Neurosci Res 2005; 81:666-676.
55. Armijo-Weingart L, Ketschek A, Sainath R, Pacheco A, Smith G M, Gallo G.

Neurotrophins induce fission of mitochondria along embryonic sensory axons. Elife 2019; 8.
56. Sahenk Z, Yalvac M E, Amornvit J, Arnold W D, Chen L, Shontz K M, Lewis S. Efficacy of exogenous pyruvate in Trembler (J) mouse model of Charcot-Marie-Tooth neuropathy. Brain and behavior 2018; 8: e01118.
57. Constantinescu C S, Farooqi N, O'Brien K, Gran B. Experimental autoimmune encephalomyelitis (EAE) as a model for multiple sclerosis (MS). Br J Pharmacol 2011; 164:1079-1106.
58. Hampton D W, Serio A, Pryce G, Al-Izki S, Franklin R J, Giovannoni G, Baker D, Chandran S. Neurodegeneration progresses despite complete elimination of clinical relapses in a mouse model of multiple sclerosis. Acta Neuropathol Commun 2013; 1:84.
59. Goswami T K, Singh M, Dhawan M, Mitra S, Emran T B, Rabaan A A, Mutair A A, Alawi Z A, Alhumaid S, Dhama K. Regulatory T cells (Tregs) and their therapeutic potential against autoimmune disorders-Advances and challenges. Hum Vaccin Immunother 2022; 18:2035117.
60. Attfield K E, Jensen L T, Kaufmann M, Friese M A, Fugger L. The immunology of multiple sclerosis. Nat Rev Immunol 2022; 22:734-750.
61. Smith M E, Eller N L, McFarland H F, Racke M K, Raine C S. Age dependence of clinical and pathological manifestations of autoimmune demyelination. Implications for multiple sclerosis. The American journal of pathology 1999; 155:1147-1161.

What is claimed:

1. A method of treating multiple sclerosis (MS) in a human subject in need thereof comprising the step of administering to the human subject a nucleic acid encoding a NT-3 polypeptide; wherein
a) the nucleic acid comprises a nucleotide sequence that is at least 90% identical to the nucleotide sequence of SEQ ID NO: 1;
b) the nucleic acid comprises the nucleotide sequence of SEQ ID NO: 1;
c) the nucleic acid comprises a nucleotide sequence encoding an amino acid sequence that is at least 90% identical to SEQ ID NO: 2; or
d) the nucleic acid comprises a nucleotide sequence encoding the amino acid sequence of SEQ ID NO: 2.

2. A method of treating an autoimmune disease in a human subject in need thereof comprising the step of administering to the human subject a nucleic acid encoding a NT-3 polypeptide; wherein
a) the nucleic acid comprises a nucleotide sequence that is at least 90% identical to the nucleotide sequence of SEQ ID NO: 1;
b) the nucleic acid comprises the nucleotide sequence of SEQ ID NO: 1;
c) the nucleic acid comprises a nucleotide sequence encoding an amino acid sequence that is at least 90% identical to SEQ ID NO: 2; or
d) the nucleic acid comprises a nucleotide sequence encoding the amino acid sequence of SEQ ID NO: 2.

3. The method of claim 2, wherein the autoimmune disease is Alopecia areata, Addison disease, Celiac disease, Crohn's disease, Ulcerative colitis, Autoimmune inflammatory myositis, Graves disease, Hashimoto thyroiditis, inflammatory bowel disease, Multiple sclerosis, Pemphigus, Pernicious anemia, Psoriasis, Reactive arthritis, Rheumatoid arthritis, Sjögren syndrome, Systemic lupus erythematosus, Type I diabetes, or Autoimmune hepatitis.

4. The method of any one of claims 1-3, wherein the nucleic acid encoding the NT-3 polypeptide is operatively linked to a muscle-specific promoter.

5. The method of claim 4, wherein the muscle-specific promoter is muscle-specific creatine kinase promoter (MCK).

6. The method of claim 5, wherein the muscle creatine kinase promoter has the nucleotide sequence set out in SEQ ID NO: 11.

7. The method of any one of claims 1-6, wherein the nucleic acid is administered using a viral vector.

8. The method of claim 7, wherein the viral vector is a recombinant adeno-associated virus (rAAV).

9. The method of claim 8, wherein the rAAV capsid serotype is AAV-1, AAV-2, AAV-3, AAV-4, AAV-5, AAV-6, AAV-7, AAV-8, AAV-9, AAV-10, AAV-11, AAV12, AAV13, Anc80, AAV-B1, AAVrh. 10, or AAVrh.74.

10. The method of claim 9, wherein the rAAV capsid serotype is AAV-1.

11. The method of any one of claims 8-10, wherein the rAAV genome sequence comprises in order from 5′ to 3′:
(i) a first AAV2 inverted terminal repeat sequence (ITR);
(ii) a muscle creatine kinase promoter/enhancer sequence set out in nucleotides 147-860 of SEQ ID NO: 3;
(iii) a nucleotide sequence encoding a human NT-3 polypeptide; and
(iv) a second AAV2 ITR sequence;
wherein the human NT-3 polypeptide has an amino acid sequence that is at least 90% identical to SEQ ID NO: 2 or is 100% identical to SEQ ID NO: 2, or is encoded by a nucleotide sequence at least 90% identical to nucleotides 1077-1850 of SEQ ID NO: 3 or 100% identical to nucleotides 1077-1850 of SEQ ID NO: 3.

12. The method of claim 11, wherein the nucleic acid sequence further comprises 3′ to said promoter/enhancer, a chimeric intron set out in nucleotides 892-1024 of SEQ ID NO: 3.

13. The method of claim 11 or 12, wherein the nucleic acid sequence further comprises 3′ to said nucleotide sequence encoding a human NT-3 polypeptide, a SV40 polyadenylation signal set out in nucleotides 1860-2059 of SEQ ID NO: 3.

14. The method of any one of claims 11-13, wherein said first ITR is set out in nucleotides 7-112 of SEQ ID NO: 3, and/or said second ITR is set out in nucleotides 2121-2248 of SEQ ID NO: 3.

15. The method of any one of claims 1-14, wherein the nucleic acid comprises the SCAAV1.tMCK.NTF3 rAAV genome that is at least 90% identical to SEQ ID NO: 9.

16. The method of any one of claims 1-14, wherein the nucleic acid comprising the SCAAV1.tMCK.NTF3 genome is set out in SEQ ID NO: 9.

17. The method of any one of claims 1-16, wherein the nucleic acid is administered at a dose that results in sustained expression of a low concentration of NT-3 polypeptide.

18. The method of any one of claims 1-17, wherein administering the nucleic acid or rAAV reduces inflammation in an organ affected by MS or an autoimmune disease in the subject.

19. The method of claim 18, wherein the organ affected by MS or an autoimmune disease is a brain, spinal cord, joints, muscles, skin, pancreas, liver, or kidney.

20. The method of claim 18, wherein the reduction in inflammation markers and inflammatory cytokines such as TNFα, IL1B, IL6, IL17, or IL22.

21. The method of any one of claims 1-20, wherein administering the nucleic acid or rAAV modulates an immune response in the subject.

22. The method of claim 21, wherein the percentage of regulatory T cells is increased in an organ affected by MS or an autoimmune disease.

23. The method of claim 22, wherein the organ affected by MS or an autoimmune disease is a lymph node, spleen, thymus, or peripheral blood.

24. The method of any one of claims 1-23, wherein administering the nucleic acid or rAAV modulates the cytokine expression in dendritic cells in the subject.

25. The method of any one of claims 1-24, wherein the nucleic acid or rAAV is administered by intramuscular injection.

26. The method of any one of claims 1-25, wherein the subject is an older adult subject.

27. A viral vector for use in the treatment of multiple sclerosis (MS) in a human subject in need thereof, wherein the composition comprises a nucleic acid encoding a NT-3 polypeptide; wherein
a) the nucleic acid comprises a nucleotide sequence that is at least 90% identical to the nucleotide sequence of SEQ ID NO: 1;
b) the nucleic acid comprises the nucleotide sequence of SEQ ID NO: 1;
c) the nucleic acid comprises a nucleotide sequence encoding an amino acid sequence that is at least 90% identical to SEQ ID NO: 2; or
d) the nucleic acid comprises a nucleotide sequence encoding the amino acid sequence of SEQ ID NO: 2.

28. A viral vector for use in treating an autoimmune disease in a human subject in need thereof; wherein the composition comprises a nucleic acid encoding a NT-3 polypeptide; wherein
a) the nucleic acid comprises a nucleotide sequence that is at least 90% identical to the nucleotide sequence of SEQ ID NO: 1;
b) the nucleic acid comprises the nucleotide sequence of SEQ ID NO: 1;
c) the nucleic acid comprises a nucleotide sequence encoding an amino acid sequence that is at least 90% identical to SEQ ID NO: 2; or
d) the nucleic acid comprises a nucleotide sequence encoding the amino acid sequence of SEQ ID NO: 2.

29. The viral vector of claim 28, wherein the autoimmune disease is Alopecia areata, Addison disease, Celiac disease, Crohn's disease, Ulcerative colitis, Autoimmune inflammatory myositis, Graves disease, Hashimoto thyroiditis, inflammatory bowel disease, Multiple sclerosis, Pemphigus, Pernicious anemia, Psoriasis, Reactive arthritis, Rheumatoid arthritis, Sjögren syndrome, Systemic lupus erythematosus, Type I diabetes, or Autoimmune hepatitis.

30. The viral vector of any one of claims 27-29, wherein the nucleic acid encoding the NT-3 polypeptide is operatively linked to a muscle-specific promoter.

31. The viral vector of claim 30, wherein the muscle-specific promoter is muscle-specific creatine kinase promoter (MCK).

32. The viral vector of claim 31, wherein the muscle creatine kinase promoter has the nucleotide sequence set out in SEQ ID NO: 11.

33. The viral vector of any one of claims 27-32, wherein the nucleic acid is administered using a viral vector.

34. The viral vector of claim 33, wherein the viral vector is a recombinant adeno-associated virus (rAAV).

35. The viral vector of claim 34, wherein the rAAV capsid serotype is AAV-1, AAV-2, AAV-3, AAV-4, AAV-5, AAV-6, AAV-7, AAV-8, AAV-9, AAV-10, AAV-11, AAV12, AAV13, Anc80, AAV-B1, AAVrh. 10, or AAVrh.74.

36. The viral vector of claim 34, wherein the rAAV capsid serotype is AAV-1.

37. The viral vector of any one of claims 34-36, wherein the rAAV genome sequence comprises in order from 5′ to 3′:
(i) a first AAV2 inverted terminal repeat sequence (ITR);
(ii) a muscle creatine kinase promoter/enhancer sequence set out in nucleotides 147-860 of SEQ ID NO: 3;
(iii) a nucleotide sequence encoding a human NT-3 polypeptide; and
(iv) a second AAV2 ITR sequence;
wherein the human NT-3 polypeptide has an amino acid sequence that is at least 90% identical to SEQ ID NO: 2 or is 100% identical to SEQ ID NO: 2, or is encoded by a nucleotide sequence at least 90% identical to nucleotides 1077-1850 of SEQ ID NO: 3 or 100% identical to nucleotides 1077-1850 of SEQ ID NO: 3.

38. The viral vector of claim 37, wherein the nucleic acid sequence further comprises 3′ to said promoter/enhancer, a chimeric intron set out in nucleotides 892-1024 of SEQ ID NO: 3.

39. The viral vector of claim 37 or 38, wherein the nucleic acid sequence further comprises 3′ to said nucleotide sequence encoding a human NT-3 polypeptide, a SV40 polyadenylation signal set out in nucleotides 1860-2059 of SEQ ID NO: 3.

40. The viral vector of any one of claims 37-39, wherein said first ITR is set out in nucleotides 7-112 of SEQ ID NO: 3, and/or said second ITR is set out in nucleotides 2121-2248 of SEQ ID NO: 3.

41. The viral vector of any one of claims 27-40, wherein the nucleic acid comprises the scAAV1.tMCK.NTF3 rAAV genome that is at least 90% identical to SEQ ID NO: 9.

42. The viral vector of any one of claims 27-40, wherein the nucleic acid comprising the scAAV1.tMCK.NTF3 genome is set out in SEQ ID NO: 9.

43. The viral vector of any one of claims 27-42, wherein the nucleic acid is administered at a dose that results in sustained expression of a low concentration of NT-3 polypeptide.

44. The viral vector of any one of claims 27-43, wherein administering the nucleic acid or rAAV reduces inflammation in an organ affected by MS or an autoimmune disease in the subject.

45. The viral vector of claim 44, wherein the organ affected by MS or an autoimmune disease is a brain, spinal cord, joints, muscles, skin, pancreas, liver, or kidney.

46. The viral vector of claim 44, wherein the reduction in inflammation markers and inflammatory cytokines such as TNFα, IL1B, IL6, IL17, or IL22.

47. The viral vector of any one of claims 27-46, wherein administering the nucleic acid or rAAV modulates an immune response in the subject.

48. The viral vector of claim 47, wherein the percentage of regulatory T cells is increased in an organ affected by MS or an autoimmune disease.

49. The viral vector of claim 48, wherein the organ affected by MS or an autoimmune disease is a lymph node, spleen, thymus, or peripheral blood.

50. The viral vector of any one of claims 27-49, wherein administering the nucleic acid or rAAV modulates the cytokine expression in dendritic cells in the subject.

51. The viral vector of any one of claims 27-50, wherein the composition is formulated for administration by intramuscular injection.

52. The viral vector of any one of claims 27-51, wherein the subject is an older adult.

53. Use of a nucleic acid encoding a NT-3 polypeptide for the preparation of a medicament for treating multiple sclerosis (MS) in a human subject in need thereof, wherein
a) the nucleic acid comprises a nucleotide sequence that is at least 90% identical to the nucleotide sequence of SEQ ID NO: 1;
b) the nucleic acid comprises the nucleotide sequence of SEQ ID NO: 1;
c) the nucleic acid comprises a nucleotide sequence encoding an amino acid sequence that is at least 90% identical to SEQ ID NO: 2; or
d) the nucleic acid comprises a nucleotide sequence encoding the amino acid sequence of SEQ ID NO: 2.

54. Use of a nucleic acid encoding a NT-3 polypeptide for the preparation of a medicament for treating an autoimmune disease in a human subject in need thereof; wherein
a) the nucleic acid comprises a nucleotide sequence that is at least 90% identical to the nucleotide sequence of SEQ ID NO: 1;
b) the nucleic acid comprises the nucleotide sequence of SEQ ID NO: 1;
c) the nucleic acid comprises a nucleotide sequence encoding an amino acid sequence that is at least 90% identical to SEQ ID NO: 2; or
d) the nucleic acid comprises a nucleotide sequence encoding the amino acid sequence of SEQ ID NO: 2.

55. The use of claim 54, wherein the autoimmune disease is Alopecia areata, Addison disease, Celiac disease, Crohn's disease, Ulcerative colitis, Autoimmune inflammatory myositis, Graves disease, Hashimoto thyroiditis, inflammatory bowel disease, Multiple sclerosis, Pemphigus, Pernicious anemia, Psoriasis, Reactive arthritis, Rheumatoid arthritis, Sjögren syndrome, Systemic lupus erythematosus, Type I diabetes, or Autoimmune hepatitis.

56. The use of any one of claims 53-55, wherein the nucleic acid encoding the NT-3 polypeptide is operatively linked to a muscle-specific promoter.

57. The use of claim 56, wherein the muscle-specific promoter is muscle-specific creatine kinase promoter (MCK).

58. The use of claim 57, wherein the muscle creatine kinase promoter has the nucleotide sequence set out in SEQ ID NO: 11.

59. The use of any one of claims 53-58, wherein the nucleic acid is administered using a viral vector.

60. The use of claim 59, wherein the viral vector is a recombinant adeno-associated virus (rAAV).

61. The use of claim 60, wherein the rAAV capsid serotype is AAV-1, AAV-2, AAV-3, AAV-4, AAV-5, AAV-6, AAV-7, AAV-8, AAV-9, AAV-10, AAV-11, AAV12, AAV13, Anc80, AAV-B1, AAVrh.10, or AAVrh.74.

62. The use of claim 60, wherein the rAAV capsid serotype is AAV-1.

63. The use of any one of claims 60-62, wherein the rAAV genome sequence comprises in order from 5′ to 3′:
(i) a first AAV2 inverted terminal repeat sequence (ITR);
(ii) a muscle creatine kinase promoter/enhancer sequence set out in nucleotides 147-860 of SEQ ID NO: 3;
(iii) a nucleotide sequence encoding a human NT-3 polypeptide; and
(iv) a second AAV2 ITR sequence;
wherein the human NT-3 polypeptide has an amino acid sequence that is at least 90% identical to SEQ ID NO: 2 or is 100% identical to SEQ ID NO: 2, or is encoded by a nucleotide sequence at least 90% identical to nucleotides 1077-1850 of SEQ ID NO: 3 or 100% identical to nucleotides 1077-1850 of SEQ ID NO: 3.

64. The use of claim 63, wherein the nucleic acid sequence further comprises 3′ to said promoter/enhancer, a chimeric intron set out in nucleotides 892-1024 of SEQ ID NO: 3.

65. The use of claim 63 or 64, wherein the nucleic acid sequence further comprises 3′ to said nucleotide sequence encoding a human NT-3 polypeptide, a SV40 polyadenylation signal set out in nucleotides 1860-2059 of SEQ ID NO: 3.

66. The use of any one of claims 63-65, wherein said first ITR is set out in nucleotides 7-112 of SEQ ID NO: 3, and/or said second ITR is set out in nucleotides 2121-2248 of SEQ ID NO: 3.

67. The use of any one of claims 53-66, wherein the nucleic acid comprises the scAAV1.tMCK.NTF3 rAAV genome that is at least 90% identical to SEQ ID NO: 9.

68. The use of any one of claims 53-66, wherein the nucleic acid comprising the scAAV1.tMCK.NTF3 genome is set out in SEQ ID NO: 9.

69. The use of any one of claims 53-68, wherein the nucleic acid is administered at a dose that results in sustained expression of a low concentration of NT-3 polypeptide.

70. The use of any one of claims 53-69, wherein administering the nucleic acid or rAAV reduces inflammation in an organ affected by MS or an autoimmune disease in the subject.

71. The use of claim 70, wherein the organ affected by MS or an autoimmune disease is a brain, spinal cord, joints, muscles, skin, pancreas, liver, or kidney.

72. The use of claim 70, wherein the reduction in inflammation markers and inflammatory cytokines such as TNFα, IL1B, IL6, IL17, or IL22.

73. The use of any one of claims 53-72, wherein administering the nucleic acid or rAAV modulates an immune response in the subject.

74. The use of claim 73, wherein the percentage of regulatory T cells is increased in an organ affected by MS or an autoimmune disease.

75. The use of claim 74, wherein the organ affected by MS or an autoimmune disease is a lymph node, spleen, thymus, or peripheral blood.

76. The use of any one of claims 53-75, wherein administering the nucleic acid or rAAV modulates the cytokine expression in dendritic cells in the subject.

77. The use of any one of claims 53-76, wherein the composition is formulated for administration by intramuscular injection.

78. The use of any one of claims 53-77, wherein the subject is an older adult.


SEQUENCE LISTING (12 sequences)

SEQ ID NO: 1 - DNA, 774 bp, Homo sapiens
  source 1..774: mol_type = genomic DNA
atgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatga

SEQ ID NO: 2 - protein, 270 aa, Homo sapiens
MVTFATILQVNKVMSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 3 - DNA, 5884 bp, synthetic construct
  source 1..5884: mol_type = other DNA; note = sc pAAV.tMCK.NTF3 plasmid genome full Sequence
cagcagctgcgcgctcgctcgctcactgaggccgcccgggcaaagcccgggcgtcgggcgacctttggtcgcccggcctcagtgagcgagcgagcgcgcagagagggagtggggttaaccaattggcggccgcaaacttgcatgccccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcacctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagcgcgtggtacctgcagggatatccaccatgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatgaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 4 - DNA, 106 bp, synthetic construct
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 5 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 6 - skipped

SEQ ID NO: 7 - DNA, 200 bp, synthetic construct
  source 1..200: mol_type = other DNA; note = POLY A sequence
ggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttc

SEQ ID NO: 8 - DNA, 128 bp, synthetic construct
  source 1..128: mol_type = other DNA; note = 3'ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 9 - DNA, 2248 bp, synthetic construct
  source 1..2248: mol_type = other DNA; note = AAV.tMCK.NTF3 genome sequence
cagcagctgcgcgctcgctcgctcactgaggccgcccgggcaaagcccgggcgtcgggcgacctttggtcgcccggcctcagtgagcgagcgagcgcgcagagagggagtggggttaaccaattggcggccgcaaacttgcatgccccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcacctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagcgcgtggtacctgcagggatatccaccatgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatgaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 10 - DNA, 668 bp, synthetic construct
  source 1..668: mol_type = other DNA; note = pBR322 Ori
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 11 - DNA, 714 bp, synthetic construct
  source 1..714: mol_type = other DNA; note = tMCK Promoter
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcac

SEQ ID NO: 12 - DNA, 861 bp, synthetic construct
  source 1..861: mol_type = other DNA; note = AMP R Sequence
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa